SEQUENCE LISTING (11 sequences)

SEQ ID NO: 1 - DNA, 1434 bp, Bacillus thuringiensis
atggccatcgatagcaagactactttgcctaggcattcacttatccatacaatcaagctgaactctaacaagaagtatggtcctggtgacatgactaacggaaatcagttcatcatctcaaagcaggagtgggctactatcggagcctacatccagactggacttggattgccagtgaacgagcagcagttgaggacacatgttaacttgtctcaggatatctcaatccctagtgatttctctcagttgtatgacgtttactgttctgataagactagtgcagagtggtggaacaagaacttgtatcctttgatcatcaagtctgctaacgacatcgcttcatacggtttcaaggtggctggtgatccttctatcaagaaggatggatatttcaagaagttgcaggacgagcttgataacatcgttgataacaactctgatgacgacgcaatcgctaaggctatcaaggatttcaaggctagatgtggtatcttgatcaaggaagctaagcagtatgaggaagctgccaagaacatcgtgacatctctcgatcagttcttacatggtgatcagaagaagttagaaggtgttatcaacatccagaagaggttgaaggaagtgcagacagctcttaaccaggctcatggagagtctagtccagctcataaggagctcttggagaaagtgaagaacttgaagacaacattagagaggactatcaaggctgagcaggaccttgagaagaaagtggagtatagcttcttgttaggaccattgttgggattcgtggtttacgagatccttgagaacactgctgttcagcatatcaagaaccagatcgatgagatcaagaagcagttggattctgctcagcatgatttggacagggacgtgaagattatcggaatgttgaacagtatcaacacagatatcgacaacctttactctcaaggacaggaagcaatcaaggttttccagaagttgcaaggaatctgggctactatcggtgctcagatcgagaaccttaggacaacatcattgcaagaggttcaggactctgatgacgctgatgagatccagatcgaactcgaggacgcttctgacgcttggttggttgtggctcaggaggctagggatttcacacttaacgcttactcaactaactctaggcagaaccttccaatcaacgttatctcagattcatgtaactgttcaacaacaaacatgacatcaaaccaatacagcaatccaacaacaaacatgacatcaaaccagtacatgatctcacatgagtacacaagcttgccaaacaacttcatgttgtcaaggaactctaacttggaatacaagtgtcctgagaacaacttcatgatctattggtacaacaactctgactggtacaacaactctgattggtacaacaaccctccataa

SEQ ID NO: 2 - protein, 1245 aa, Bacillus thuringiensis
MATINELYPVPYNVLAHPIKEVDDPYSWSNLLKGIQEGWEEWGKTGQKKLFEDHLTIAWNLYKTGKLDYFALTKASISLIGFIPGAEAAVPFINMFVDFVWPKLFGANTEGKDQQLFNAIMDAVNKMVDNKFLSYNLSTLNKTIEGLQGNLGLFQNAIQVAICQGSTPERVNFDQNCTPCNPNQPCKDDLDRVASRFDTANSQFTQHLPEFKNPWSDENSTQEFKRTSVELTLPMYTTVATLHLLLYEGYIEFMTKWNFHNEQYLNNLKVELQQLIHSYSETVRTSFLQFLPTLNNRSKSSVNAYNRYVRNMTVNCLDIAATWPTFDTHNYHQGGKLDLTRIILSDTAGPIEEYTTGDKTSGPEHSNITPNNILDTPSPTYQHSFVSVDSIVYSRKELQQLDIATYSTNNSNNCHPYGLRLSYTDGSRYDYGDNQPDFTTSNNNYCHNSYTAPITLVNARHLYNAKGSLQNVESLVVSTVNGGSGSCICDAWINYLRPPQTSKNESRPDQKINVLYPITETVNKGTGGNLGVISAYVPMELVPENVIGDVNADTKLPLTQLKGFPFEKYGSEYNNRGISLVREWINGNNAVKLSNSQSVGIQITNQTKQKYEIRCRYASKGDNNVYFNVDLSENPFRNSISFGSTESSVVGVQGENGKYILKSITTVEIPAGSFYVHITNQGSSDLFLDRIEFVPKIQFQFCDNNNLHCDCNNPVDTDCTFCCVCTSLTDCDCNNPRGLDCTLCCQVENQLPSFVTLTDLQNITTQVNALVASSEHDTLATDVSDYEIEEVVLKVDALSGEVFGKEKKALRKLVNHTKRLSKARNLLIGGNFDNLDAWYRGRNVVNVSDHELFKSDHVLLPPPTLYSSYMFQKVEESKLKANTRYTVSGFIAHAEDLEIVVSRYGQEVKKVVQVPYGEAFPLTSRGAICCPPRSTSNGKPADPHFFSYSIDVGTLDVEANPGIELGLRIVERTGMARVSNLEIREDRPLKKNELRNVQRAARNWRTAYDQERAEVTALIQPVLNQINALYENEDWNGAIRSGVSYHDLEAIVLPTLPKLNHWFMSDMLGEQGSILAQFQEALDRAYTQLEESTILHNGHFTTDAANWTIEGDAHHAILEDGRRVLRLPDWSSSVSQTIEIENFDPDKEYQLVFHAQGEGTVSLQHGEEGEYVETHPHKSANFTTSHRQGVTFETNKVTVEITSEDGEFLVDHIALVEAPLPTDDQSSDGNTTSNTNSNTSMNNNQ

SEQ ID NO: 3 - protein, 475 aa, Bacillus thuringiensis
MIIDSKTTLPRHSLIHTIKLNSNKKYGPGDMTNGNQFIISKQEWATIGAYIQTGLGLPVNEQQLRTHVNLSQDISIPSDFSQLYDVYCSDKTSAEWWNKNLYPLIIKSANDIASYGFKVAGDPSIKKDGYFKKLQDELDNIVDNNSDDDAIAKAIKDFKARCGILIKEAKQYEEAAKNIVTSLDQFLHGDQKKLEGVINIQKRLKEVQTALNQAHGESSPAHKELLEKVKNLKTTLERTIKAEQDLEKKVEYSFLLGPLLGFVVYEILENTAVQHIKNQIDEIKKQLDSAQHDLDRDVKIIGMLNSINTDIDNLYSQGQEAIKVFQKLQGIWATIGAQIENLRTTSLQEVQDSDDADEIQIELEDASDAWLVVAQEARDFTLNAYSTNSRQNLPINVISDSCNCSTTNMTSNQYSNPTTNMTSNQYMISHEYTSLPNNFMLSRNSNLEYKCPENNFMIYWYNNSDWYNNSDWYNN

SEQ ID NO: 4 - DNA, 41 bp, Artificial Sequence
  A synthetic oligonucleotide
gacgatgagtctaccaggatccttttttttttttttttttt

SEQ ID NO: 5 - DNA, 22 bp, Artificial Sequence
  A synthetic oligonucleotide
gacgatgagtctaccaggatcc

SEQ ID NO: 6 - DNA, 35 bp, Artificial Sequence
  A synthetic oligonucleotide
cggtcgacaaaatggccatcgatagcaagactact

SEQ ID NO: 7 - DNA, 2112 bp, Bacillus thuringiensis
gtcgacaaaatggcaaccatcaacgagctgtacccagtgccctacaatgtactcgctcacccaatcaaggaagtagacgatccttatagttggtctaacttgctgaagggaatccaagagggctgggaggaatggggaaagacgggccagaagaagctctttgaggaccatttgacaattgcgtggaacctgtacaagaccggaaagttagattacttcgcactcactaaggcttcaatcagtctgattggcttcatcccaggagccgaggctgcagtccccttcatcaatatgtttgttgacttcgtttggccaaagttgttcggcgccaacacagaaggaaaggatcaacagctcttcaacgcaatcatggacgctgtgaataagatggtcgataacaagtttctgtcctacaacttgtccacgctcaacaaaaccatagagggcctccaaggaaacttgggcttattccagaatgccatccaggttgctatctgtcaaggatctactcccgagcgcgtcaatttcgaccagaactgcacaccatgcaatcctaaccagccatgcaaggatgacctcgatagagtggccagtcgattcgacaccgctaactcacagtttacccagcatcttcctgaattcaagaatccatggagcgatgagaactccacgcaagaattcaagagaactagcgtcgaactcacacttcctatgtacaccactgtggccacactccaccttctcttgtacgaggggtacatagagttcatgaccaaatggaacttccacaacgaacagtacctcaacaaccttaaggttgagttgcagcaactcatccactcatacagcgagactgtgaggacatcttttcttcagttcttgccaaccctcaataaccggtcaaaatctagcgtcaacgcctacaatcgttacgtgaggaacatgactgttaactgtcttgacatcgctgcaacatggccgaccttcgatactcacaattaccaccaaggtgggaagttggacctcacacggatcatcctttcagataccgccggtccaatcgaggagtacactactggagacaagacctctggtcccgagcactccaacatcactcctaacaatatcttggatacaccgtcccccacctatcagcattctttcgtcagcgtggactccattgtctattctaggaaggagctccagcaacttgatatcgctacttacagcacaaacaactccaataactgccatccttacgggttgagactctcttacaccgacggtagcagatacgattacggagacaaccagcctgatttcactacatccaataacaactattgtcacaattcttacaccgcaccgattactcttgttaacgccagacacttgtacaacgctaaaggaagcctacagaatgtggaatccctcgtggtctctacagttaacgggggtagcgggtcctgcatttgcgacgcatggattaactatttgcgtcccccacaaacctctaagaacgagtccaggcctgaccagaaaatcaacgtgctatacccaattactgagacagtgaacaagggtaccgggggcaatctgggagtaatctcggcttacgttcctatggagttggtgccagagaacgtcattggtgacgtgaacgcagacaccaagttgcctctgacacaattgaaggggttcccattcgagaagtatggtagtgaatacaataaccgtggaatctcgctcgttagagaatggatcaatggaaacaacgcagtcaagctgtccaattcgcagtccgtcgggatacagattaccaatcagactaaacagaagtacgagatccgttgccgatatgcttcaaagggggataacaatgtttacttcaacgtggacttgtctgagaacccctttcgcaatagtatttcattcgggtccacagaatcatctgtggttggcgtccaaggggagaacggcaagtacatcctcaagagtattaccactgtggagatcccagccggatcattctatgtccacattacaaaccagggctcctcagacctgttcttggataggatcgaattcgttcctaagattcagtaaggatcc

SEQ ID NO: 8 - DNA, 3561 bp, Bacillus thuringiensis
atggattgtaatttacaatcacaacaaaatattccttataatgtattagcaataccagtatctaatgttaatgcgttggttgatacagctggagatttaaaaaaagcatgggaagaatttcaaaaaactggttctttttcattaacagctttacaacaaggattttctgcctcacaaggaggagcattcaattatttaacattattacaatcaggaatatcattagctggttcttttgtccctggaggtacttttgtagcacccattgttaatatggttattggttggttatggccacataaaaacaagacagcggatacagaaaatttaataaaattaattgatgaagaaattcaaaaacaattaaacaaagccttattagaccaagatagaaacaattggacctcttttttagaaagtatatttgatacttcagctacagtaagtaatgcaattatagatgcacagtggtcaggtactgtagatactacaaatagacaacaaaaaactccaacaacatcagattatctaaatgttgttggaaaatttgattcagcggattcttcaattataactaatgaaaatcaaataatgaatggcaactttgacgtagctgcagcaccctattttgttataggagcaacattacgtctttcattatatcaatcttatattaaattttgtaatagttggattgatgcagttggatttagtacaaatgatgctaatacacaaaaagctaatttagctcgtacgaaattaactatgcgtactacaattaatgaatatacacaaagagttatgaaagtttttaaagattccaagaatatgcctacaataggtactaataaatttagtgttgatgcttataatgtatatgttaaaggaatgacattaaatgttttagatatggtagcaatatggtcttcattatatccaaatgattatacttcacaaacagccatagaacaaacacgtgtcactttttcaaatatggttggacaagaagaaggtacagatggaaccctaaaaatttacaatacttttgattctcttagttatcaacatagcctaatacctaataataatgttaatttaatttcttattatactgatgaattgcaaaatctagaattagcagtatatactcctaaaggtggaagtggatacgcttatccttatggatttattttaaattatgcaaacagcaactacaaatatggtgataatgatccaacaggcaaaccattaaataaacaagatggacctatacaacaaataaatgcagcaactcaaaacagtaaatatctagatggagaaacaataaatggaataggggcatccttacctggttattgtactacaggatgttcagcaacagaacaaccttttagttgtacttctactgctaatagctataaagcaagctgtaatccttcagatactaatcaaaaaattaatgctttatatgcttttacacaaactaatgtaaagggaagcacggggaaattaggagtactggcaagtcttgttccatatgatttaaatcctaaaaatgtatttggtgaattagattcagatacaaataatgttatcttaaaaggaattcctgcagaaaaagggtattttcctaataatgcgcgacctactgttgtaaaagaatggattaatggtgcaagtgctgtaccattttattcaggaaatactttatttatgacggctacgaatttaacagctactcaatataaaattagaatacgttatgcaaatccaaattcagatactcaaatcggtgtactaattacgcaaaatggttctcaaatttccaatagtaatctaacactttatagtactactgattcaagtatgagtagtaatttaccacaaaatgtatatgtcacaggggaaaatggaaattatacacttctagatttatatagtactactaatgttttatcaacaggagatattacattaaaacttacaggaggaaatcaaaaaatatttattgatcgaatagaatttattcctactatgcctgtacctgctcctactaataacactaataacaataacggcgataacggcaataacaatcccccacaccacggttgtgcaatagctggtacacaacaactttgttctggaccacctaagtttgaacaagtaagtgatttagaaaaaattacaacgcaagtatatatgttattcaaatcttcttcgtatgaagaattagctctaaaagtttctagctatcaaattaatcaagtggcattgaaagttatggcactatctgatgaaaagttttgtgaagaaaaaagattgttacgaaaattagtcaataaagcaaaccaattactagaagcacgtaacttactagtaggtggaaattttgaaacaactcaaaattgggtacttggaacaaatgcttatataaattatgattcgtttttatttaatggaaattatttatccttacaaccagcaagtggatttttcacatcttatgcttatcaaaaaatagatgagtcaacattaaaaccatatacacgatataaagtttctggattcattgggcaaagtaatcaagtagaacttattatttctcgttatggaaaagaaattgataaaatattaaatgttccatatgcagggcctcttcctattactgctgatgcatcgataacttgttgtgcaccagaaatagaccaatgtgatggggggcaatctgattctcatttcttcaactatagcatcgatgtaggtgcacttcacccagaattaaaccctggcattgaaattggtcttaaaattgtgcaatcaaatggttatataacaattagtaatctagaaattattgaagaacgtccacttacagaaatggaaattcaagcagtcaatcgaaaagatcaaaaatggaaaagagaaaaacttctagaatgtgcaagtgttagtgaacttttacaaccaatcattaatcaaatcgattcattgttcaaagatgcaaactggtataatgatattcttcctcatgtcacatatcaaactctaaaaaatattatagtacccgatttaccaaaattaaaacattggttcatagatcatctcccaggtgaatatcatgaaattgaacaaaaaatgaaagaagctctaaaacatgcatttacacaattagacgagaaaaatttaatccacaatggtcactttgcaactaacttaatagattggcaagtagaaggtgatgctcgaatgaaagtattagaaaataatgctttggcattacaactttccaattgggattctagtgtttcacaatctattgatatattagaatttgatgaagataaagcatataaacttcgcgtatatgctcaaggaagcggaacaatccaatttggaaactgtgaagatgaagccatccaatttaatacaaactcattcgtatataaagaaaaaataatctatttcgataccccatcaattaacttacacatacaatcagaaggttctgaattcgttgtaagtagtatcgacctcgttgaattatcagacgacgaataa

SEQ ID NO: 9 - protein, 1186 aa, Bacillus thuringiensis
MDCNLQSQQNIPYNVLAIPVSNVNALVDTAGDLKKAWEEFQKTGSFSLTALQQGFSASQGGAFNYLTLLQSGISLAGSFVPGGTFVAPIVNMVIGWLWPHKNKTADTENLIKLIDEEIQKQLNKALLDQDRNNWTSFLESIFDTSATVSNAIIDAQWSGTVDTTNRQQKTPTTSDYLNVVGKFDSADSSIITNENQIMNGNFDVAAAPYFVIGATLRLSLYQSYIKFCNSWIDAVGFSTNDANTQKANLARTKLTMRTTINEYTQRVMKVFKDSKNMPTIGTNKFSVDAYNVYVKGMTLNVLDMVAIWSSLYPNDYTSQTAIEQTRVTFSNMVGQEEGTDGTLKIYNTFDSLSYQHSLIPNNNVNLISYYTDELQNLELAVYTPKGGSGYAYPYGFILNYANSNYKYGDNDPTGKPLNKQDGPIQQINAATQNSKYLDGETINGIGASLPGYCTTGCSATEQPFSCTSTANSYKASCNPSDTNQKINALYAFTQTNVKGSTGKLGVLASLVPYDLNPKNVFGELDSDTNNVILKGIPAEKGYFPNNARPTVVKEWINGASAVPFYSGNTLFMTATNLTATQYKIRIRYANPNSDTQIGVLITQNGSQISNSNLTLYSTTDSSMSSNLPQNVYVTGENGNYTLLDLYSTTNVLSTGDITLKLTGGNQKIFIDRIEFIPTMPVPAPTNNTNNNNGDNGNNNPPHHGCAIAGTQQLCSGPPKFEQVSDLEKITTQVYMLFKSSSYEELALKVSSYQINQVALKVMALSDEKFCEEKRLLRKLVNKANQLLEARNLLVGGNFETTQNWVLGTNAYINYDSFLFNGNYLSLQPASGFFTSYAYQKIDESTLKPYTRYKVSGFIGQSNQVELIISRYGKEIDKILNVPYAGPLPITADASITCCAPEIDQCDGGQSDSHFFNYSIDVGALHPELNPGIEIGLKIVQSNGYITISNLEIIEERPLTEMEIQAVNRKDQKWKREKLLECASVSELLQPIINQIDSLFKDANWYNDILPHVTYQTLKNIIVPDLPKLKHWFIDHLPGEYHEIEQKMKEALKHAFTQLDEKNLIHNGHFATNLIDWQVEGDARMKVLENNALALQLSNWDSSVSQSIDILEFDEDKAYKLRVYAQGSGTIQFGNCEDEAIQFNTNSFVYKEKIIYFDTPSINLHIQSEGSEFVVSSIDLVELSDDE

SEQ ID NO: 10 - DNA, 3504 bp, Bacillus thuringiensis
atgacaaatccaactatactatatcctagttaccataatgtattagctcatccgattagattagattctttttttgatccatttgtagagacatttaaggatttaaaaggggcttgggaagaattcggaaaaacgggatatatggaccccttaaaacaacaccttcaaatcgcatgggatactagtcaaaatggaacagtggattatttagcattaacaaaagcatctatatctctcataggtttaattcctggtgcagacgctgtagtcccttttattaatatgtttgtagactttatttttccgaaattatttggaagaggttctcaacaaaatgctcaagctcaatttttcgaactaatcatagaaaaagttaaagaacttgttgatgaagattttagaaactttacccttaataatctactcaattaccttgatggtatgcaaacagccttatcacatttccaaaacgatgtacaaattgctatttgtcaaggagaacaaccaggacttatgctagatcaaacaccaacggcttgtactcctactacagaccatttaatttctgtaagagaatcttttaaagatgctcgaactacaattgaaacagctttaccacattttaaaaatcctatgctatccacaaatgataacactccagattttaatagcgacactgtcttattaacattaccaatgtatacaacaggagcgactttaaatcttatattacatcaagggtatattcaattcgcagaaagatggaaatctgtaaattatgatgaaagttttataaatcaaacaaaagttgatttgcaacgtcgtattcaggactattctactactgtatctaccacttttgaaaaattcaaacctactctaaatccatcaaataaagaatctgttaataagtataatagatatgttcgttccatgactcttcaatctttagacattgctgcaacatggcctactttagataatgttaattacccttccaatgtagatattcaattggatcaaactcgcttagtattttcagatgttgcaggaccttgggaaggtaatgataatataacttcgaatattatagatgtattaacaccaataaatacagggataggatttcaagaaagttcagatcttagaaaattcacttatccacgaatagaattacaaagcatgcaattccatggacaatatgtaaactcaaaaagtgtagaacattgttatagcgatggtcttaaattaaattataaaaataaaactataactgcaggtgtaagtaatattgatgaaagtaatcaaaataataaacataactatggtcctgtaataaatagtcctattactgatatcaacgtaaattcccaaaattctcaatatttagatttaaattcagtcatggtaaatggtggtcaaaaagtaaccgggtgttcaccacttagttcaaatggtaattctaataatgctgctttacctaatcaaaaaataaatgttatttattcagtacaatcaaatgataaaccagaaaaacatgcagacacttatagaaaatggggatatatgagcagtcatattccttatgatcttgttccagaaaatgtaattggagatatagatccggatactaaacaaccgtcattgcttcttaaagggtttccggcagaaaaaggatatggtgactcaattgcatatgtatcagaacctttaaatggtgcgaatgcagttaaacttacttcatatcaagttctccaaatggaagttacaaatcaaacaactcaaaaatatcgtattcgcatacgttatgctacaggtggagatacagctgcttctatatggtttcatattattggtccatctggaaatgatttaacaaacgaaggccataacttctctagtgtatcttctagaaataaaatgtttgttcagggtaataacggaaaatatgtattgaacatccttacagattcaatagaattaccatcaggacaacaaactattcttattcaaaatactaattctcaagatctttttttagatcgtattgaatttatttctctcccttctacttctactcctacttctactaattttgtagaacctgaatcattagaaaagatcataaaccaagttaatcaattatttagctcctcatctcaaactgaattggctcacactgtaagcgattataaaattgatcaagtagtgctaaaagtaaatgccttatccgacgatgtatttggtgtagagaaaaaagcattacgtaaacttgtgaatcaggccaaacaactcagtaaagcacgaaatgtattggtcggtggaaactttgaaaaaggtcatgaatgggcactaagccgtgaagcaacaatggtcgcaaatcatgagttattcaaaggggatcatttattattaccaccaccaaccctatatccatcgtatgcatatcaaaaaattgatgaatcgaaattaaaatccaatacacgttatactgtttccggctttattgcgcaaagtgaacatctagaagtcgttgtgtctcgatacgggaaagaagtacatgacatgttagatatcccgtatgaagaagccttaccaatttcttctgatgagagtccaaattgttgcaaaccagctgcttgtcagtgttcatcttgtgatggtagtcaatcagattctcatttctttagctatagtatcgatgttggttccctacaatcagatgtaaatctcggcattgaattcggtcttcgtattgcgaaaccaaacggatttgcgaaaatcagtaatctagaaattaaagaagatcgtccattaacagaaaaagaaatcaaaaaagtacaacgtaaagaacaaaaatggaaaaaagcatttaaccaagaacaagccgaagtagcgacaacactccaaccaacgttagatcaaatcaatgctttgtatcaaaatgaagattggaacggttccgttcacccggccagtgactatcaacatctgtccgctgttgttgtaccaacgttaccaaaacaaagacattggtttatggagggtcgagaaggcgaacatgttgttctgacgcaacaattccaacaagcattggatcgtgcgttccaacaaatcgaagaacaaaacttaatccacaatggtaatttggcgaatggattaacagattggactgtcacaggagatgcacaacttacgatctttgacgaagatccagtattagaactagcgcattgggatgcaagtatctctcaaaccattgaaattatggattttgaaggaagacacagaatacaaactgcgtgtacgtggaaaaggcaaaggaacagttaccgttcaacatggaggaagagattagaaacgatgacattcaatacaacgagttttacaacacaagaacaaaccttctacttcgaaggagatacagtggacgtacatgttcaatcagagaataacacattcctgatagatagtgtggaactcattgaaatcatagaagagtaa

SEQ ID NO: 11 - protein, 1167 aa, Bacillus thuringiensis
MTNPTILYPSYHNVLAHPIRLDSFFDPFVETFKDLKGAWEEFGKTGYMDPLKQHLQIAWDTSQNGTVDYLALTKASISLIGLIPGADAVVPFINMFVDFIFPKLFGRGSQQNAQAQFFELIIEKVKELVDEDFRNFTLNNLLNYLDGMQTALSHFQNDVQIAICQGEQPGLMLDQTPTACTPTTDHLISVRESFKDARTTIETALPHFKNPMLSTNDNTPDFNSDTVLLTLPMYTTGATLNLILHQGYIQFAERWKSVNYDESFINQTKVDLQRRIQDYSTTVSTTFEKFKPTLNPSNKESVNKYNRYVRSMTLQSLDIAATWPTLDNVNYPSNVDIQLDQTRLVFSDVAGPWEGNDNITSNIIDVLTPINTGIGFQESSDLRKFTYPRIELQSMQFHGQYVNSKSVEHCYSDGLKLNYKNKTITAGVSNIDESNQNNKHNYGPVINSPITDINVNSQNSQYLDLNSVMVNGGQKVTGCSPLSSNGNSNNAALPNQKINVIYSVQSNDKPEKHADTYRKWGYMSSHIPYDLVPENVIGDIDPDTKQPSLLLKGFPAEKGYGDSIAYVSEPLNGANAVKLTSYQVLQMEVTNQTTQKYRIRIRYATGGDTAASIWFHIIGPSGNDLTNEGHNFSSVSSRNKMFVQGNNGKYVLNILTDSIELPSGQQTILIQNTNSQDLFLDRIEFISLPSTSTPTSTNFVEPESLEKIINQVNQLFSSSSQTELAHTVSDYKIDQVVLKVNALSDDVFGVEKKALRKLVNQAKQLSKARNVLVGGNFEKGHEWALSREATMVANHELFKGDHLLLPPPTLYPSYAYQKIDESKLKSNTRYTVSGFIAQSEHLEVVVSRYGKEVHDMLDIPYEEALPISSDESPNCCKPAACQCSSCDGSQSDSHFFSYSIDVGSLQSDVNLGIEFGLRIAKPNGFAKISNLEIKEDRPLTEKEIKKVQRKEQKWKKAFNQEQAEVATTLQPTLDQINALYQNEDWNGSVHPASDYQHLSAVVVPTLPKQRHWFMEGREGEHVVLTQQFQQALDRAFQQIEEQNLIHNGNLANGLTDWTVTGDAQLTIFDEDPVLELAHWDASISQTIEIMDFEGRHRIQTACTWKRQRNSYRSTWRKRLETMTFNTTSFTTQEQTFYFEGDTVDVHVQSENNTFLIDSVELIEIIEE

(19) United States
(12) Patent Application Publication
(10) Pub. No.: US 2010/0024075 A1
(43) Pub. Date: Jan. 28, 2010

(54) Method for Controlling Plant-Parasitic Nematode Infections in Plants

(76) Inventors: Raffi V. Aroian, San Diego, CA (US); Xiang-Oian Li, Chesterfield, MO (US)

Correspondence Address: SCHWEGMAN, LUNDBERG & WOESSNER, P.A., P.O. BOX 2938, MINNEAPOLIS, MN, 55402, US

(21) Appl. No.: 12/085,472

(22) PCT Filed: Nov. 22, 2006
(86) PCT No.: PCT/US2006/045106

§ 371 Date: Mar. 17, 2009

Related U.S. Application Data
(60) Provisional application No. 60/739,866, filed on Nov. 23, 2005.
(60) Provisional application No. 60/804,250, filed on Jun. 8, 2006.

(51) Int. Cl. A01H 5/00; A61K 38/16; C07H 21/00

(52) U.S. Cl. 800/301; 514/12; 536/23.1

(57) ABSTRACT

The invention provides compositions having Bacillus thuringiensis Crystal (Cry) proteins including transgenic plants expressing those proteins, and methods of using those proteins to prevent, inhibit or treat parasitic infections in plants and vertebrates.

### CROSS-REFERENCE TO RELATED APPLICATIONS

The present application claims the benefit of the filing date of U.S. application Ser. No. 60/739,866, filed on Nov. 23, 2005, and of U.S. application Ser. No. 60/804,250, filed on Jun. 8, 2006, the disclosures of which are incorporated by reference herein.

### STATEMENT OF GOVERNMENT RIGHTS

This invention was made at least in part with a grant from the U.S. Government (grant R01 AI056189-01A2 from the National Institutes of Health). The U.S. Government has certain rights in the invention.

### BACKGROUND

Plant parasitic nematodes (PPNs) that infect the roots of crops cause an estimated annual economic loss of $77 billion worldwide (Sasser et al., 1987). They also play an important role in limiting food production in developing countries. For example, migratory PPNs seriously impact the production of bananas and plantain in Africa (Speijer et al., 1997). The root knot nematode, Meloidogyne incognita, is one of the most important PPNs. M. incognita is able to infect more than 2000 plant species (Jung et al., 1999). It can be a limiting factor in cotton and peanut production and causes greater yield losses in cotton than any other pathogen in the United States (Blasingame et al., 2003).

PPNs are difficult pests to control without the use of chemical nematicides, and the availability of these nematicides is decreasing due to high toxicity to humans and the environment (Lirshitz et al., 2000; El-Alfy et al., 2002). Furthermore under the Montreal Protocol of 1987, one of the main chemicals used to control PPNs, methyl bromide, is being phased out of use around the world due to its harm to ozone layer (Ristaino et al., 1997).

It is thus imperative that alternative strategies for controlling PPNs be developed.

### SUMMARY OF THE INVENTION

Crystal (Cry) proteins produced by the soil bacterium Bacillus thuringiensis (Bt) are harmless to vertebrates but highly toxic to a variety of invertebrate species. For instance, B. thuringiensis Cry proteins in the Cry5 and Cry6 subclades intoxicate free-living nematodes.

The invention provides compositions and methods to inhibit or treat nematode infections in plants. To prepare transgenic plants expressing Bt toxin genes, the nucleotide sequence of Cry genes, e.g., the Cry5B or Cry6A genes, may be altered from the wild-type gene in order to allow the expression of the gene in plants, e.g., plant roots. In one embodiment, the changes included replacement of codons with plant codons, removal of premature polyadenylation sites, removal of potential splice sites, removal of rare plant codons, addition of two proline residues at the C-terminus to increase protein stability, addition of a plant promoter to increase expression, or a combination thereof. Moreover, the inclusion of a plant intron, e.g., from the UBQ10 gene, inserted into the Cry6A gene was found to increase expression.

In one embodiment, a codon-modified cry6A gene driven by the constitutive cauliflower mosaic virus 35S promoter was introduced into tomato roots via Agrobacterium rhizogenes. These roots were then infected with the root-knot nematode, Meloidogyne incognita. Characterization and quantitation of M. incognita infections on control versus Cry6A-expressing roots demonstrated that M. incognita ingests the 54 kDa Cry6A protein and that the nematode is intoxicated by Cry6A, as indicated by up to a 4-fold reduction in progeny production. The inhibition of nematode infection by Cry6A protein was surprising, as it was thought that the expression of those toxins in plants might not inhibit nematodes because endoparasitic nematodes, especially sedentary ones, would not ingest such large proteins. Further, expression of a Cry6A gene truncated to nucleotide 1158 in transgenic tomato controlled infections of M. incognita in transgenic tomato roots. One effect of Cry6A expression was to reduce the production of eggs from mothers infecting the plant, which in turn decreases the propagation of the infection in the field. Transgenic Cry5B or Cry6A plants represent a major step forward in agricultural control of parasitic nematodes.

As also described herein, a series of mutations in a truncated Cry6A gene that yield variant Cry6A proteins increased its potency against C. elegans (a free-living nematode) and allowed the truncated toxin to inhibit Cry6A resistant nematodes. These mutations include: (1) Q52L and T235A; (2) K170N; (3) K99N; (4) K19N, K192E, K205R, K249R and Q319L; (6) L138V; (7) G121S; (8) Q62R and D147G, and (9) I164T and E237G. As also described herein, a series of mutations in a truncated Cry5B gene that yield valiant Cry5B proteins increased its potency against C. elegans, e.g., (1) S407C and (2) S663P.

In one embodiment, the substitutions in full length or truncated Cry proteins are conservative substitutions. In one embodiment, the substitutions are nonconservative substitutions. In one embodiment, lysine (K) and/or glutamine (Q) residues are substituted. Thus, both truncated and substituted Cry sequences, as well as wild-type Cry sequences, may be employed in the compositions and methods of the invention.

As further described herein, the in vitro and in vivo antihelminthic activity of purified recombinant Cry5B against the hookworm parasite Ancylostoma ceylanicum, a bloodfeeding gastrointestinal nematode for which humans are fully permissive hosts, was characterized. Using in vitro egg hatch and larval development assays, Cry5B was found to be highly toxic to early stage hookworm larvae. Exposure of adult A. ceylanicum to Cry5B was also associated with significant toxicity, including a substantial reduction in egg excretion by adult female worms. In order to demonstrate therapeutic efficacy in vivo, hamsters infected with A. ceylanicum were treated with three consecutive daily oral doses of Cry5B (3×1 mg), the benzimidazole antihelminthic mebendazole, or buffer. Compared to control (buffer-treated) animals, infected hamsters that received Cry5B showed statistically significant improvements in growth and blood hemoglobin levels, as well as reduced worm burdens, and a near cure of the infection, that were comparable to the mebendazole treated animals. These data demonstrate that nematicidal Bt Cry toxins are highly active in vivo against a globally significant nematode parasite.

Therefore, the invention also provides compositions and methods useful in the treatment of intestinal animal parasitic nematode diseases in veterinary medicine and human medicine. In one embodiment, the invention provides compositions comprising Bt crystal proteins to inhibit or treat hookworm and other intestinal parasitic helminth (nematode) infections in the intestines of vertebrate animals, e.g., mammals such as feline, rodent, canine, bovine, equine, swine, caprine, and primates, e.g., humans. The compositions also control the population of infectious helminths in the environment. The use of the compounds is cost effective and the administration may be in the form of food, e.g., transgenic food, making it possible to delivery the therapy as part of the diet. For instance, parasitic infection of humans or nonhuman vertebrates may be prevented or treated by consumption of transgenic fruits and vegetables that express Cry proteins, variants thereof, or truncations thereof. Accordingly, the invention provides a method to inhibit or treat animal parasitic helminth infection. The method includes administering to an animal, e.g., a mammal, an effective amount of a Cry protein. In one embodiment, the crystal protein which is administered is a crystal protein that kills animal parasitic worms.

Cry21A and Cry14A are also nematicidal and part of the same clade of proteins as Cry5B and so may be useful in the compositions and methods of the invention. Based on resistance profiles in C. elegans, resistance to Cry21A and Cry5B is via a different pathway. Moreover, Cry14A and Cry21A are toxic to the same nematodes that Cry5A intoxicates including the rodent nematode Nippostrongylus. Therefore, a combination of crystal proteins may be employed in a therapeutic treatment, e.g., Cry5B and Cry21A together, so as to avoid resistance. In one embodiment, to treat intestinal parasitic nematode infections (like hookworm) in humans and other vertebrates, the vertebrate is administered a tablet/pill form of a Bt crystal toxin, e.g., Cry14A, Cry21A, or Cry5B, or a combination thereof. In another embodiment, the crystal protein is expressed in a food plant and that plant or a product prepared from that plant is ingested by an infected human or nonhuman vertebrate. In addition, since Bt crystal proteins are often dumped in the environment to control insect pests, nematicidal crystal proteins may also be used by application to the environment to kill and/or control nematode pest populations before they infect their targets.

In one embodiment, the invention provides for a method of treatment, either prophylactic or therapeutic treatment, of a subject or a patient population exposed to or at risk of exposure to a parasitic infection by a worm.

The use of Bt crystal proteins is advantageous in that those proteins are safe and non-toxic to vertebrates and unlike currently used anti-nematode compounds, nematode pests are not resistance to Bt crystal proteins. Moreover, crystal proteins may provide an alternative therapy for animal parasitic nematode infections in those cases where resistance to currently used compounds has occurred. Alternatively, co-therapy of crystal protein and currently used compounds, e.g., albendazole, mebendazole, levaminsole, or ivermectin, may provide an advantage as such therapy would be predicted to be resistance-free for a long time.

The invention further provides constructs (vectors) having nucleic acid encoding a truncated Cry protein or variants of a full-length or truncated Cry protein, and synthetic Cry genes, e.g., synthetic Cry5 and Cry6 genes, having changes including removal of premature polyadenylation sites, removal of potential splice sites, replacement of codons with codons preferred in plants, removal of rare plant codons, addition of two proline residues at the C-terminus to increase protein stability, and/or addition of a plant promoter sequence to increase expression. Also provided are isolated Cry5, Cry6, Cry14, and Cry21 truncated proteins and variants of a full-length or truncated Cry proteins, host cells having the constructs, and methods of preparing plants having the constructs.

### BRIEF DESCRIPTION OF THE DRAWING

FIG. 1. Vectors for tomato root and Arabidopsis transformation.

FIG. 2. Expression of Cry6A genes in tomato hairy root lines. (A) Plasmid map. A plant codon-friendly version cat 6A gene (1425 nt) was synthesized by assembling 70-90-mer oligonucleotides and then subcloned into the pBIN-JIT binary vector driven by the CaMV 35S promoter (P) with duplicated enhancer and terminated by the 35S terminator (T) for plant transformation. (B) Cry6A expression as evidenced by a Western blot was probed with polyclonal anti-Cry6A antibody. Left lanes: E. coli produced Cry6A loading controls. Right lanes: total soluble protein from hairy root lines (1 μg protein/lane). A hairy root line transformed with pBIN-JIT vector (V) was used as a negative control. Three Cry6A hairy root lines (6A-1, -13 and -15) out of a total of 20 lines are representatively shown. (C). Reverse transcription-PCR analysis and Northern blot. The first strand of cDNA was synthesized by M-MLV reverse transcriptase with oligd(T) linked to an adapter sequence. This cDNA was then PCR amplified with 3′ 5′ primers. Sequencing of these RT-PCR products demonstrates that the cry6A gene was transcribed with correct coding sequence and terminator sequence. A total of 10 μg RNA was electrophoresed in a 1.2% formaldehyde-agarose gel and probed with P³²-labeled full length cry6A. (D) The lengths of transformed roots starting from 2 cm-long root tips were measured on 6 consecutive days. (E) The average dry weight of 8 roots per line 45 days after initial subculture of 2 cm root tips. Means followed by the same letter were not significantly different at P<0.05. (F) Gail percentage in control and transgenic lines.

FIG. 3. Intoxication of M. incognita by Cry6A. (A) The left and right panels show M. incognita galls that developed respectively on vector-transformed and Cry6A-transformed (6A-13) lines. The red arrows point to galls induced by M. incognita infection. The galls are smaller in the Cry6A expressing line. (B) Total egg mass number produced on the 6 lines normalized to that of vector control. V=vector control, GFP=GFP expressing line, 6A-1=non-Cry6A expressing line, 6Atr-6=truncated Cry6A expressing line, 6A-13 and 6A-15=Cry6A expressing lines. (C) Total eggs (progeny) produced on the 6 lines normalized to that of vector control. Means followed by the same letter were not significantly different at P<0.05.

FIG. 4. Uptake of Cry6A by M. incognita. (A) A representative of a 35 day old adult M. incognita female dissected from vector control tomato hairy roots. (B) Western blot of total protein extracted from 40 adult M. incognita dissected from vector control or Cry6A expressing tomato hairy roots and probed with affinity-purified anti-Cry6A antibody. The non-specific bands (slightly above the Cry6A band) present in both control and Cry6A lines indicate equal loading of proteins.

FIG. 5A. Expression of Cry6A 1158 in transgenic tomato and Arabidopsis.

FIG. 5B. Expression of Cry6A 1425 in transgenic tomato and Arabidopsis.

FIG. 5C. Expression of Cry6A 1425 with an intron in transgenic Arabidopsis.

FIG. 6. Top panel: All panels show representative sections of plates grown 5 days at 25° C. starting with 20 L4-staged worms. (A) This plate well was spread with vector-only (non-toxic) E. coli. Two generations of worms depleted the bacterial lawn, and the worms were starved. (B) The plate was spread with Cry5B (unmutated) E. coli. The worms grew slowly. (C) The plate was spread with one of the hypertoxic clones (S407C). The worms were sicker and smaller than in the unmutated Cry5B well (panel B). This clone was about 2× more potent than Cry5B well (panel B) against C. elegans. (D) The hypertoxic clone in panel C in which the point mutation was reverted back to wild type to demonstrate that this mutation alone is responsible for the hyperactive toxin phenotype. The animals were comparable to those in the unmutated well (panel B). The S407 mutation does not affect the level of toxin expressed in E. coli. Lower panel: The percent worms alive after 6 days at 25° C. at a given concentration of purified Cry5B. Each data point represents the average from 3 independent trials with 20 worms per trial per concentration.

FIG. 7. Ancylostoma ceylanicum extracts contain Cry5B-binding glycolipids. Upper phase glycolipids were extracted from mixed-stage C. elegans (Ce) and A. ceylanicum (Ac) adults and separated by thin-layer chromatography. The separated glycolipids were then subjected to a Cry5B overlay in the presence of 80 mM glucose (Glc) (left lanes) or 80 mM galactose (Gal) (right lanes). Cry5B bound to glycolipids from both nematodes in a galactose-dependent fashion.

FIG. 8. Effects of Cry5B on A. ceylanicum adults in vitro. (A) Purified Cry5B. Cry5B was purified as described and separated by SDS-PAGE (lane 3; 6 μg). (B) Exposure to Cry5B impairs motility of adult hookworms. Groups of 10 adult A. ceylanicum worms were cultured in increasing concentrations of purified Cry5B toxin. Motility was monitored at times indicated, and data represent the mean values (+/−standard error) of triplicate wells containing each concentration of toxin. Statistically significant (p<0.001) difference in motility were observed between control worms (0 μg/ml) and worms cultured in 50 or 200 μg/ml Cry5B toxin throughout the observation period (24-120 hours). Statistically significant differences (p<0.001) between control worms (0 μg/ml) and the 5 μg/ml toxin group were detected from 42-120 hours in culture. (C) Cry5B toxin reduces A. ceylanicum egg excretion. Adult female hookworms were maintained for 24 hours in the presence of increasing concentrations of purified Cry5B toxin. Experimental groups consisting of 4 replicate wells (3 worms/well) at each concentration of toxin. Values represent mean number of eggs (+/−standard error) counted in each well.

FIG. 9. Effects of Cry5B on A. ceylanicum larvae. (A) Cry5B toxin impairs motility of early stage (L1/L2) hookworm larvae. A. ceylanicum eggs were allowed to hatch over 48 hours in the presence of increasing concentrations of purified Cry5B toxin. Experimental groups consisting of 4 replicate wells containing larvae (mean of 20-46/well) at each concentration of toxin. Values represent mean percent of motile larvae (+/−standard error) counted in each well. P values represent statistical comparisons between each group and the control worms (no toxin). (B) Cry5B toxicity in early larval (L1/L2) development. Representative photomicrographs (200× magnification) show stunted growth and loss of integrity of the tegument and internal structures in A. ceylanicum larvae exposed to purified Cry5B.

FIG. 10. Cry5B treatment reduces clinical sequelae of hookworm infection as measured by weight gain (top panels) and blood hemoglobin (bottom panels). Hamsters were infected with 150 A. ceylanicum L3 on day 0 and treated with Cry5B (left panels) or mebendazole (right panels) on days 14, 15, and 16 as indicated by open arrows. All values are means +/− standard error. (*) indicates statistical significance versus the infected control group.

FIG. 11. Cry5B treatment reduces fecal egg excretion (left A) and intestinal hookworm burden (right B) in infected hamsters. A) Fecal samples from infected animals were collected at times indicated and hookworm eggs quantified as described in the methods. All values are means +/−standard error. (*) indicates statistical significance (p<0.05) versus the infected control group. B) Individual worm burdens are indicated by closed circles and means of each group by horizontal bars. Brackets indicate statistical comparisons between groups, with P values indicated.

FIG. 12. Nematodes on plates with Cry5B (A) or Cry14A (B) truncated proteins, or control.

FIG. 13. Summary of Cry21A truncations and toxicities.

FIG. 14. Amino acid sequences of wild-type Cry5B and Cry6A (SEQ ID NO:2 and SEQ ID NO:3) and nucleic acid sequences encoding truncated Cry5B and Cry6A sequences for plant expression (SEQ ID NO:7 and SEQ ID NO: 1, respectively).

FIG. 15. Nucleotide and amino acid sequence of Cry14A (SEQ ID NO:8 and SEQ ID NO:9) and Cry21A (SEQ ID NO:10 and SEQ ID NO:11).

### DETAILED DESCRIPTION OF THE INVENTION

### Definitions

By “truncated,” when referring to a Bt toxin protein (crystal protein) is meant a Bt toxin protein that is not full-length but retains at least 10%, 50% or more, e.g., at least 70%, 80%, 90% or more, the toxic activity of a corresponding full-length Bt toxin protein.

A “variant” polypeptide is a polypeptide with one or more substitutions, e.g., no more than 10 substitutions, for instance 1 to 5 substitutions, or substitutions at 10% or fewer of the residues, relative to a corresponding wild-type polypeptide or truncated version thereof. The variant polypeptide or the truncated wild-type or variant polypeptide has at least 10%, e.g., at least 20%, 50% or more, for instance, 100% more of the activity, e.g., toxic activity, of the corresponding wild-type polypeptide or truncated version. Conservative substitutions include substitutions within the following groups: glycine, alanine, threonine, valine, isoleucine, leucine; aspartic acid, glutamic acid, asparagine, glutamine; serine, cysteine; lysine, arginine; aspartic acid, glutamic acid; serine, threonine; asparagine, glutamine; phenylalanine, tyrosine.

Two amino acid or nucleic acid sequences are homologous if there is a partial or complete identity between their sequences. For example, 85% homology means that 85% of the amino acids or nucleotides are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

As used herein, the term “sequence identity” means that two sequences are identical over the window of comparison. The term “percentage of sequence identity” means that two sequences are identical over the window of comparison. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the window of comparison (i.e., the window size), and multiplying the result by 100 to yield the percentage of sequence identity.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

By “substantially pure” is meant a nucleic acid, polypeptide, or other molecule that has been separated from the components that naturally accompany it. Typically, the polypeptide is substantially pure when it is at least 60%, 70%, 80%, 90% 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

By “cloning” is meant the use of in vitro techniques to insert a particular gene or other DNA sequence into a vector molecule. In order to successfully clone a desired gene, it is necessary to employ methods for generating DNA fragments, for joining the fragments to vector molecules, for introducing the composite DNA molecule into a host cell in which it can replicate, and for selecting the clone having the target gene from amongst the recipient host cells.

By “vector” is meant a DNA molecule, usually derived from a plasmid or bacteriophage, into which fragments of DNA may be inserted or cloned. A vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

By “operably linked” is meant that a nucleic acid molecule and one or more regulatory sequences (e.g., a promoter) are connected in such a way as to permit expression and/or secretion of the product (i.e., a polypeptide) of the nucleic acid molecule when the appropriate molecules (e.g., transcriptional activator proteins) are bound to the regulatory sequences.

By “transgene” is meant any piece of nucleic acid that is inserted by artifice into a cell, or an ancestor thereof, and becomes part of the genome of the organism which develops from that cell. Such a transgene may include a gene which is partly or entirely heterologous (i.e., foreign) to the transgenic organism, or may represent a gene homologous to an endogenous gene of the organism.

A “transgenic plant” refers to any plant in which one or more of the cells of the plant contain heterologous nucleic acid introduced by way of human intervention, such as transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within the chromosome, or it may be extra-chromosomally replicating DNA. In the typical transgenic plants described herein, the transgene causes cells to express a recombinant form of one of the subject polypeptide.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65 C, or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. See, e.g., F. Ausubel et al., Current Protocols in Molecular Biology, John Wiley & Sons, New York, N.Y., 1998, hereby incorporated by reference.

The term “heritable” refers to the fact that the nucleic acid molecule is capable of transmission through a complete sexual cycle of a plant, i.e., it is passed from one plant through its gametes to progeny plants in the same manner as occurs in normal plants, or the nucleic acid can be transmitted via asexual propagation of cuttings or shoots.

The term “exogenous,” as used herein in reference to a nucleic acid molecule and a transgenic plant, means a nucleic acid molecule that is not native to the plant or that is present in the genome in other than its native association. An exogenous nucleic acid molecule can have a naturally occurring or non-naturally occurring nucleotide sequence and can be orthologous or heterologous to the plant species into which it is introduced. The term “plant expression vector,” as used herein, is a self-replicating nucleic acid molecule that provides a means to transfer an exogenous nucleic acid molecule into a plant host cell and to express the molecule therein. Plant expression vectors encompass vectors suitable for Agrobacterium-mediated transformation, including binary and cointegrating vectors, as well as vectors for physical transformation.

### Bt Proteins and Genes

Bacillus thuringiensis (Bt) crystal (Cry) proteins are the most widely used biologically-produced insecticides in the world (Whalen et al., 2003). Bt is a soil bacterium that produces large crystalline inclusions upon sporulation. These crystals contain one or several Cry proteins that are highly toxic to invertebrates (Naimor et al., 2001; Wei et al., 2003). For decades, Cry proteins have been applied in large quantities to agricultural fields and the environment in order to kill both insects (caterpillars and beetles) that eat plants and insect vectors (mosquitoes and black flies) that transmit viruses and helminthes (Federici et al., 2005). More recently, transgenic crops expressing Bt Cry proteins have been found to provide strong protection against caterpillars (REF). In the year 2005, over 26 million hectares (mha) of Bt crops were planted, including 8.5 mha of Bt cotton, accounting for about 24% of all the cotton grown in the world (James, 2005). The tremendous success of this natural resource is due to multiple factors, including high efficacy, absence of toxicity of Cry proteins towards mammals and other vertebrates, and the ability to produce Cry proteins cheaply and in massive quantities. Bt Cry proteins confer substantial benefit to the environment and farm worker safety by reducing applications of toxic chemical insecticides that adversely affect farm workers, other non-target animals, and the environment (Qaim et al, Hossain et al, Carrier et al).

Three Cry proteins, Cry5B, Cry14A, and Cry21A, that are toxic to both free-living nematodes (roundworms) and the free-living stage of one parasitic nematode, have been identified and characterized (Wei et al., 2003). Vertebrate-safe Cry proteins could be used to treat both human and veterinary nematode infections. In particular, soil transmitted nematodes (STNs) are a legitimate class of therapeutic targets for Bt Cry proteins, since Bt is a soil bacterium that potentially interacts with and/or confronts these pathogens in their shared natural environment. Globally, STN infections represent a major cause of morbidity in developing countries, with an estimated burden of human disease comparable that of malaria or tuberculosis (Savoili et al., 2004; Chan, 1997; Crompton et al., 2003; deSilva et al., 2003; Molyneux et al., 2005). In addition to human disease, animal nematode infections are of major veterinary significance, resulting in millions of dollars of lost revenue effecting industries that provide products and food from livestock, including cattle and sheep (Coles, 2005; Coles et al., 2005a; Chandrawathani et al., 2003; Mertz et al., 2005; Von Sampson et al., 2005; Wolstenholme et al., 2004; McKellar et al., 2004). In light of well-founded concerns about the emergence of antihelminthic resistance, there exists a pressing need to develop new, safe, and inexpensive agents for the treatment of human and veterinary nematode infections of global significance (Wolsten et al., 2004; Geerts et al., 2001; Albonico, 2003; Albonico et al., 2004; Reynoldson et al., 1997; DeClercq et al., 1997).

As described herein, members of the Cry5 and Cry6 subfamilies of Bt toxins were tested for nematicidal activity. Testing was performed on Cry5 toxin subfamily members Cry5Aa, Cry5B, Cry12A, Cry13A, Cry14A, and Cry21A. Cry5Ab and Cry5Ac, which are about 96% identical to Cry5Aa, are also members of the Cry5 subfamily. The Cry6 subfamily of toxins includes Cry6A and Cry6B, which are about 50% identical to each other but unrelated to the other known Bt toxins. Both Cry6 subfamily members were tested for nematicidal activity.

The strategy of truncating Bt toxin proteins is generally applicable to enhance the effectiveness of other Bt toxins. The usefulness of Bt toxins for controlling nematodes may be limited by the protein size that nematodes can ingest, particularly plant parasitic nematodes. Typically, these nematodes poorly ingest proteins larger than about 40 kD. Thus, the effectiveness of any particular Bt toxin may be limited by size exclusion of proteins that nematodes take in and so should be small enough to be readily absorbed by the nematode gut while retaining nematicidal activity. There are other compelling reasons to produce a toxin truncated from the full length version. A truncated toxin would be easier to express in plants. Producing a truncated toxin will also alleviate the requirement that the target nematode have the proper proteases present to correctly process full length protoxin (which is inactive) to a truncated, active toxin form. Thus, a truncated toxin will be immediately available for intoxication independent of whether the proper protease processing enzymes are present in the nematode target. Truncated toxin may also express at a higher level in a plant since truncated toxins are soluble and less likely to form insoluble inclusions in the cell expressing them, which could be toxic to the cell or which could make the toxin fold incorrectly. Accordingly, it is desirable to produce truncated Bt toxin fragments. Moreover, fragments of certain Bt toxins were tested and shown to retain nematicidal activity and have improved biological properties.

Sources of nucleotide sequences from which the present nucleic acid molecules encoding a Cry protein, a variant thereof, a truncated form thereof, or the nucleic acid complement thereof, include previously isolated or previously modified, as well as newly isolated, Bacillus Cry sequences. Thus, DNA or RNA encoding a wild-type Cry sequence may be isolated by methods known in the art. Other sources of the DNA molecules of the invention include libraries, e.g., mutagenized or genomic libraries.

Nucleic acid molecules encoding amino acid sequence variants, truncated versions, or both, of a Cry protein are prepared by a variety of methods known in the art. These methods include, but are not limited to, isolation from a natural source (in the case of naturally occurring amino acid sequence variants) or preparation by, for example, oligonucleotide-mediated (or site-directed) mutagenesis, PCR mutagenesis, and cassette mutagenesis of an earlier prepared variant or a non-variant version of protein. Moreover, the invention includes synthetic nucleic acid molecules where nucleotides are modified to include codons preferred in a particular organism, remove codons rarely used in a particular organism, or remove sequences that may inhibit transcription or RNA processing and the like.

In one embodiment, a Bt toxin gene is modified for expression in plants. For instance, one or more prokaryotic codons are replaced with plant codons, and regulatory sequences may optionally be introduced or removed (see, e.g., U.S. Pat. Nos. 5,952,547 and 6,169,232).

For instance, sequences may be modified including removal of premature polyadenylation sites, removal of potential splice sites, removal of rare plant codons, addition of two proline residues at the C-terminus to increase protein stability, and addition of a plant promoter sequence to increase expression. In one embodiment, an optimized Cry gene (a plant version of cry6A sequence) has:

(SEQ ID NO: 1)

ATGgcCATCGATAGCAAGACTACTTTGCCTAGGCATTCACTTATCCATAC
AATCAAGcTGAACTCTAACAAGAAGTATGGTCCTGGTGACATGACTAACG
GAAATCAGTTCATCATCTCAAAGCAGGAGTGGGCTACTATCGGAGCCTAC
ATCCAGACTGGACTTGGaTTGCCAGTGAACGAGCAGCAGTTGAGGACACA
TGTTAACTTGTCTCAGGATATCTCAATCCCTAGTGATTTCTCTCAGTTGT
ATGACGTTTACTGTTCTGATAAGACTAGTGCaGAGTGGTGGAACAAGAAC
TTGTATCCTTTGATCATCAAGTCTGCTAACGACATCGCTTCATACGGTTT
CAAGGTGGCTGGTGATCCTTCTATCAAGAAGGATGGATATTTCAAGAAGT
TGCAGGACGAGCTTGATAACATCGTTGATAACAACTCTGATGACGACGCA
ATCGCTAAGGCTATCAAGGATTTCAAGGCTAGATGTGGTATCTTGATCAA
GGAAGCTAAGCAGTATGAGGAAGCTGCCAAGAACATCGTGACATCTCTCG
ATCAGTTCTTACATGGTGATCAGAAGAAGTTAGAAGGTGTTATCAACATC
CAGAAGAGGTTGAAGGAAGTGCAGACAGCTCTTAACCAGGCTCATGGaGA
GTCTAGTCCAGCTGATAAGGAGCTCTTGGAGAAaGTGAAGAACTTGAAGA
CAACATTAGAGAGGACTATCAAGGCTGAGCAGGACCTTGAGAAGAAaGTG
GAGTATAGCTTCTTGTTAGGACCATTGTTGGGATTCGTGGTTTACGAGAT
CCTTGAGAACACTGCTGTTCAGCATATCAAGAACCAGATCGATGAGATCA
AGAAGCAGTTGGATTCTGCTCAGCATGATTTGGACAGGGACGTGAAGATt
ATCGGAATGTTGAACAGTATCAACACAGATATCGACAACCTTTACTCTCA
AGGACAGGAAGCAATCAAGGTTTTCCAGAAGTTGCAAGGaATCTGGGCTA
CTATCGGTGCTCAGATCGAGAACCTTAGGACAACATCATTGCAAGAGGTT
CAGGACTCTGATGACGCTGATGAGATCCAGATCGAACTCGAGGACGCTTC
TGACGCTTGGTTGGTTGTGGCTCAGGAGGCTAGGGATTTCACACTTAACG
CTTACTCAACTAACTCTAGGCAGAACCTTCCAATCAACGTTATCTCAGAT
TCATGTAACTGTTCAACAACAAACATGACATCAAACCAATACAGCAATCC
AACAACAAACATGACATCAAACCAGTACATGATCTCACATGAGTACACAA
GCTTGCCAAACAACTTCATGTTGTCAAGGAACTCTAACTTGGAATACAAG
TGTCCTGAGAACAACTTCATGATCTATTGGTACAACAACTCTGACTGGTA
CAACAACTCTGATTGGTACAACAACcctccataa.

To prepare expression cassettes for transformation herein, the recombinant DNA may be circular or linear, double-stranded or single-stranded. Generally, the DNA is in the form of chimeric DNA, such as plasmid DNA, that can also contain coding regions flanked by control sequences which promote the expression of the preselected DNA present. As used herein, “chimeric” means that a vector comprises DNA from at least two different species, or comprises DNA from the same species, which is linked or associated in a manner which does not occur in the “native” or wild type of the species.

### Exemplary Parasites

The present invention relates to the control of parasitic worms, e.g., nematodes and platyhelminths, using crystal proteins from Bacillus and their derivatives. Parasitic worms within the scope of the invention include but are not limited to those in Class Adenophorea, e.g., Order Mononchida, Family Plectidae, and Order Stichosomida, Family Mermithidae and Tetradonematidae; Class Secementea, e.g., Order Rhabditida, Family Carabonematidae, Cephalobidae, Chambersiellidae, Heterorhabditidae, Oxyuridae, Panagrolaimidae, Rhabditidae, Steinemematidae, Syrphonematidae, Syrphonematidae, or Thelastomatidae; Order Spirurida, Family Filariidae, Onchocercidae, Physalopteridae, Syngamidae, Spiruridae, Subuluridae, or Thelaziidae; Order Diplogasterida, Family Diplogasteridae; and Order Tylenchida, Family Allantonematidae, Aphelenchidae, Aphelenchoididae, Entaphelenchidae, Fergusobiidae, Phaenopsitylenchidae, Sphaerulariidae, Anguinidae, Dolichodoridae, Belonolaimidae, Pratylenchidae, Hoplolamidae, Heteroderidae, Criconematidae, Tylenchulidae or Tylenehidae. In one embodiment, the parasite is from Class Secernentea, Order Ascaridida, Family Ascarididae; Class Adenophorea, Order Trichurida, Family Trichuridae; Class Secernentea, Order Strongylida, Family Ancylostomatidae (ancylostomidae) or Trichostrongylidae; or Class Secernentea, Order Spirurida, Family Dracunculidae, Filariidae, or Onchocercidae.

In one embodiment, the parasite is a helminth. Helminths within the scope of the invention include but are not limited to those from Phylum Annelida, Class Polychaetae, Class Myzostomida, Class Clitellata, Subclass Hirudinea, Order Gnathobdellidae, Order Rhynchobdellidae; Phylum Platyhelminthes (Flatworms), Class Turbellaria, Class Monogenea, Order Monopisthocotylea, Order Polyopisthocotylea, Class Trematoda, Subclass Aspidogasrea, Subclass Digenea; Super Order Anepitheliocystida, Order Strigeatida, Family Schistosomatidae, Subfamily Schistosomatinae, Genus Schistosoina, Order Echinostomatida, Family Fasciolidae, Family Paramphistomatidae, Family Echinostomatidae; Super Order Epitheliocystida, Order Plagiorchiida, Family Dicrocoeliidae, Family Troglotreinatidae, Order Opisthorchiida, Family Heterophyidae, Family Opisthorchiidae, Class Cestoda, Subclass Cestodaria, Subclass Eucestoda, Order Pseudophyllidea, Family Diphyllobothriidae, Order Cyclophyllidea, Family Taeniidae, Family Hymenolepididae, Family Dilepididae, Family Mesocestoididae, Order Tetraphyllidea, Order Proteocephalata, or Order Spatheobothridea. For example, Cry proteins with the scope of the invention may be employed to prevent, inhibit or treat Roundworm, Whipworm, Hookworm, Schistosome, or Trematodes.

### Cells and Transgenic Plants Expressing Bt Toxins and Uses Thereof

Recent evidence suggests that antihelmintic resistance is on the rise in parasites of pigs, sheep, horses, cattle, and humans. Particularly in animals, resistance can be induced by underdosing. The most common reasons for underdosing are incomplete drug delivery or inaccurate estimation of body weight. Thus, in addition to new classes of antihelmentic (e.g., nematicidal) compounds, more efficient and effective methods for administering antihelmentic compounds to animals. Accordingly, transgenic plants expressing the nematicidal Bt toxins and fragments described herein can be used as a food source for both humans and animals. Delivering antihelmentic compounds in the food supply using transgenic plants has several advantages. First, transgenic plants expressing a desirable amount of the Bt toxin or fragment can be substituted for or mixed with non-transgenic plants during farming. This strategy is particularly useful in geographic regions where parasitic nematode infections are endemic or where the availability of medical treatment is scarce. Second, delivery of nematicidal compounds to animals using transgenic plants obviates the need for antihelmentic veterinary care and herd management. Because food intake is correlated with body weight and is self-regulating, the problem of accurately estimate body weight for dosing and effectively delivering the therapeutic formulation is also overcome. Third, since the farmers in first or third world countries would be planting the crop in any event, having the crop make the therapeutic nematicidal agent would remove the need to establish any significant new infrastructure to administer the therapy (e.g., no clinics need be set up, no vaccines need be delivered to an area or administered). Fourth, given the superb tack record of Bt crystal proteins in showing no toxicity towards vertebrates when expressed in foods, this method of delivery would be predicted to be exceptionally safe to the infected vertebrate.

The expression of a Bt toxin or fragment in plants is achieved by introducing into a plant a DNA sequence encoding the desired Bt toxin or fragment, operably linked to a constitutively active promoter. Plants within the scope of the invention include but are not limited to those in the following families: Butomaceae, Alismataceae, Hydrocharitaceae, Juncaginaceae, Potamogetonaceae, Ruppiaceae, Zannichelliaceae, Zosteraceae, Araceae, Lemnaceae, Juncadeae, Cyperaceae, Gramineae/Poaceae, Sparganiaceae, Typhaceae, Liliaceae, Iridaceae, Orchidaceae, Nymphaeaceae, Ceratophyllaceae, Ranunculaceae, Papaveraceae, Fumariaceae, Ulmaceae, Cannabinaceae, Urticaceae, Myricaceae, Fagaceae, Betaulaceae, Aizoaceae, Chenopodiaceae, Portulacaceae, Caryophyllaceae, Polygonaceae, Plumbaginaceae, Elatinaceae, Guttiferae/Hypericaceae/Clusiaceae, Malvaceae, Sarraceniaceae, Droseraceae, Violaceae, Cucurbitaceae, Salicaceae, Cruciferae/Brassicaceae, Resedaceae, Empetraceae, Ericaceae, Pyrolaceae, Monotropaceae, Prinulaceae, Grossulariaceae, Crassulaceae, Saxifragaceae, Rosaceae, Leguminosae/Fabaceae, Elaeagnaceae, Halogaraceae, Lythraceae, Onagraceae, Viscaceae, Celastraceae, Aquifoliaceae, Euphorbiaceae, Rhamnaceae, Linaceae, Polygalaceae, Aceraceae, Oxalidaceae, Geraniaceae, Balsaminaceae, Araliaceae, Umbelliferae/Apiaceae, Gentianaceae, Apocynaceae, Solanaceae, Convolvulaceae, Cuscutaceae, Menyanthaceae, Boraginaceae, Lamiaceae, Hippuridaceae, Callitrichaceae, Plantaginaceae, Buddlejaceae, Oleaceae, Scrophulariaceac, Orobanchaceae, Lentibulariaceae, Campanulaceae, Rubiaceae, Caprifoliaceae, Adoxaceae, Valerianaceae, Dipsacaceae and Compositae/Asteraceae.

A number of vectors suitable for stable transfection of plant cells or for the establishment of transgenic plants are available. See, for example, Plant Molecular Biology: Essential Techniques, Jones et al., eds., John Wiley & Son, Ltd., 1997. Plant expression vectors include (1) a nucleotide sequence encoding a Bt toxin or fragment under the transcriptional control of 5′ and 3′ regulatory sequences, and (2) a dominant selectable marker. Such plant expression vectors also typically contain a transcription initiation start site, a ribosome binding site, an RNA processing signal, a transcription termination site, and/or a polyadenylation signal. In addition, expression of bacterial genes, including Bt crystal toxin genes, in plants requires modification of nucleic acids to optimize codon usage for plants. Bacterial genes have a high A-T content that often contains cryptic plant polyadenylation and splicing sites and non-optimal codon usage. Before expression in a plant, bacterial genes need to be resynthesized at the nucleotide level to encode the same protein but with codon usage (and lack of cryptic polyadenylation/splice sites) compatible with plant expression.

An example of a useful plant promoter which could be used to express a Bt toxin gene according to this invention is a caulimovirus promoter such as the cauliflower mosaic virus (CaMV) 35S promoter. These promoters confer high levels of expression in most plant tissues, and the activity of these promoters is not dependent on virally encoded proteins. The CaMV 35S promoter is also highly active in monocots. Other useful promoters include, for example, the nopaline synthase promoter and the octopine synthase promoter. Furthermore, it may be desirable to produce the Bt toxin only in an appropriate plant tissue (i.e., the edible portion) or at a specific developmental time (i.e., prior to harvest). There are a variety of promoters suitable for these applications including promoters responsible for (1) heat-regulated gene expression (see, e.g., Callis et al., 1998), (2) light-regulation gene expression (e.g., the pea rbcS-3A promoter, Kuhlemeier et al., 1989), (3) hormone-regulated gene expression (e.g., the abscisic acid responsive sequences from the Em gene of wheat, Marcotte et al., 1989), (4) organ-specific gene expression (e.g., tuber-specific storage protein gene, Roshal et al., 1987); the 23 kDa zein gene from maize, Schemthaner et al., 1988); or the French bean β-phaseolin gene, Bustos et al., 1989).

Plant expression vectors may also, optionally, include RNA processing signals (e.g., introns) that are important for efficient RNA synthesis and accumulation. The location of the RNA splice sequences can influence the level of transgene expression in plants. The processing signal may be positioned upstream or downstream of the Bt toxin coding sequence.

The expression vector may also include regulatory control regions that are generally present in the 3′ regions of plant genes. For example, a 3′ terminator region may be included in the expression vector to increase stability of the mRNA. Suitable terminators include, for example, the PI-II terminator region of potato, or terminators derived from the octopine or nopaline synthase signals.

The plant expression vector also typically contains a dominant selectable marker gene used to identify the cells that have become transformed. Useful selectable marker genes for plant systems include genes encoding antibiotic resistance genes, for example, those encoding resistance to hygromycin, kanamycin, bleomycin, G418, streptomycin, or spectinomycin. Genes encoding herbicide resistance may also be used as selectable markers; useful herbicide resistance genes include the bar gene encoding the enzyme phosphinothricin acetyltransferase.

Upon construction of the plant expression vector, several standard methods are known for introduction of the recombinant genetic material into the host plant for the generation of a transgenic plant. These methods include, for example, Agrobacterium-mediated transformation (A. tumefaciens or A. rhizogenes), particle delivery systems, micro injection protocols, polyethylene glycol (PEG) procedures, and liposome-mediated DNA uptake.

The present invention generally includes steps directed to introducing an isolated and purified DNA segment or sequence into a recipient cell to create a transformed cell. Those cells may include bacterial cells, e.g., expression of a truncated Cry5B protein in bacteria, vertebrate cells, e.g., rodent cells such as CHO cells, or plant cells. It is most likely that not all recipient cells receiving DNA segments or sequences will result in a transformed cell wherein the DNA is stably integrated into the genome and/or expressed. Some may show only initial and transient gene expression. However, certain cells from virtually any dicot or monocot species may be stably transformed, and these cells regenerated into transgenic plants.

Cells of the plant tissue source are preferably embryogenic cells or cell-lines that can regenerate fertile transgenic plants and/or seeds. The cells can be derived from either monocotyledons or dicotyledons. Suitable examples of plant species include wheat, rice, Arabidopsis, tobacco, maize, soybean, and the like.

Transformation of the cells of the plant tissue source can be conducted by any one of a number of methods known to those of skill in the art. Examples are: agitation of cells with DNA in the presence of metal or ceramic whiskers (U.S. Pat. No. 5,302,523); transformation by direct DNA transfer into plant cells by electroporation (U.S. Pat. No. 5,384,253 and U.S. Pat. No. 5,472,869; Dekeyser et al., 1990); direct DNA transfer to plant cells by PEG precipitation (Hayashimoto et al., 1990); direct DNA transfer to plant cells by microprojectile bombardment (McCabe et al., 1988); Gordon-Kamm et al., 1990); U.S. Pat. No. 5,489,520; U.S. Pat. No. 5,538,877; and U.S. Pat. No. 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction. See also, R. Chasan (1992).

One method for dicot transformation is via infection of plant cells with Agrobacterium tumefaciens using the leaf-disk protocol (Horsch et al., 1985). Methods for transformation of monocotyledonous plants utilizing Agrobacterium tumefaciens have been described by Hiei et al. (U.S. Pat. No. 5,591,616) and Saito et al. (European Patent No. 0 672 752).

An expression cassette of the invention can be introduced by methods of transformation, for example, methods which are especially effective for monocots, including, but not limited to, microprojectile bombardment of immature embryos or embryogenic callus cells, or by electroporation of embryogenic calluses.

The choice of plant tissue source for transformation will depend on the nature of the host plant and the transformation protocol. The tissue source is selected and transformed so that it retains the ability to regenerate whole, fertile plants following transformation, i.e., contains totipotent cells.

The transformation is carried out under conditions directed to the plant tissue of choice. The plant cells or tissue are exposed to the DNA carrying the isolated and purified DNA sequences for an effective period of time. This may range from a less-than-one-second pulse of electricity for electroporation to a 2-3 day co-cultivation in the presence of plasmid-bearing Agrobacterium cells. Buffers and media used will also vary with the plant tissue source and transformation protocol. Many transformation protocols employ a feeder layer of suspension culture cells (tobacco or Black Mexican Sweet corn, for example) on the surface of solid media plates, separated by a sterile filter disk from the plant cells or tissues being transformed.

### Production and Characterization of Stable Transgenic Plants

After effecting delivery of an isolated and purified DNA segment or sequence to recipient cells by any of the methods discussed above, the next steps may include identifying the transformed cells for further culturing and plant regeneration. As mentioned above, in order to improve the ability to identify transformants, one may desire to employ a selectable or screenable marker gene as, or in addition to, the expressible isolated and purified DNA segment or sequence. In this case, one would then generally assay the potentially transformed cell population by exposing the cells to a selective agent or agents, or one would screen the cells for the desired marker gene trait.

Mature plants are then obtained from cell lines that are known to express the trait. If possible, the regenerated plants are self pollinated. In addition, pollen obtained from the regenerated plants is crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants in order to introgress the isolated and purified DNA segment into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced isolated and purified DNA segment, the plant is self-pollinated at least once in order to produce a homozygous backcross converted inbred containing the isolated and purified DNA segment. Progeny of these plants are true breeding.

Alternatively, seed from transformed plants regenerated from transformed tissue cultures is grown in the field and self-pollinated to generate true breeding plants. Progenies from these plants become true breeding lines which are evaluated for a desired phenotype or trait.

Upon the identification of the superior performance of transgenic plants, the parent selections are advanced and inbred lines are produced through conventional breeding techniques. Hybrid plants having one or more parents containing the isolated and purified DNA segment are tested in commercial testing and evaluation programs and performance documented.

A. Characterization

To confirm the presence of the isolated and purified DNA segment(s) or “transgene(s)” in the regenerating plants, a variety of assays may be performed. Such assays include, for example, “molecular biological” assays well known to those of skill in the art, such as Southern and Northern blotting, RT-PCR and PCR; “biochemical” assays, such as detecting the presence of a protein product, e.g., by immunological means (ELISAs and Western blots) or by enzymatic function; plant part assays, such as stomatal aperture assays; and also, by analyzing the phenotype of the whole regenerated plant, e.g., for drought resistance.

1. DNA Integration RNA Expression and Inheritance

Genomic DNA may be isolated from callus cell lines or any plant parts to determine the presence of the isolated and purified DNA segment through the use of techniques well known to those skilled in the art. Note that intact sequences will not always be present, presumably due to rearrangement or deletion of sequences in the cell.

The presence of DNA elements introduced through the methods of this invention may be determined by polymerase chain reaction (PCR). Using this technique discreet fragments of DNA are amplified and detected by gel electrophoresis. This type of analysis permits one to determine whether an isolated and purified DNA segment is present in a stable transformant, but does not prove integration of the introduced isolated and purified DNA segment into the host cell genome. In addition, it is not possible using PCR techniques to determine whether transformants have exogenous genes introduced into different sites in the genome, i.e., whether transformants are of independent origin. It is contemplated that using PCR techniques it would be possible to clone fragments of the host genomic DNA adjacent to an introduced isolated and purified DNA segment.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced isolated and purified DNA segments in high molecular weight DNA, i.e., confirm that the introduced isolated and purified DNA segment has been integrated into the host cell genome. The technique of Southern hybridization provides information that is obtained using PCR, e.g., the presence of an isolated and purified DNA segment, but also demonstrates integration into the genome and characterizes each individual transformant.

It is contemplated that using the techniques of dot or slot blot hybridization which are modifications of Southern hybridization techniques one could obtain the same information that is derived from PCR, e.g., the presence of an isolated and purified DNA segment. However, it is well known in the art that dot or slot blot hybridization may produce misleading results, as probe may be non-specifically bound by high molecular weight DNA.

Both PCR and Southern hybridization techniques can be used to demonstrate transmission of an isolated and purified DNA segment to progeny. In most instances the characteristic Southern hybridization pattern for a given transformant will segregate in progeny as one or more Mendelian genes (Spencer et al., 1992; Laursen et al., 1994) indicating stable inheritance of the gene.

Whereas DNA analysis techniques may be conducted using DNA isolated from any part of a plant, RNA may only be expressed in particular cells or tissue types and hence it will be necessary to prepare RNA for analysis from these tissues. PCR techniques may also be used for detection and quantitation of RNA produced from introduced isolated and purified DNA segments. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and will only demonstrate the presence or absence of an RNA species.

2. Gene Expression

While Southern blotting and PCR may be used to detect the isolated and purified DNA segment in question, they do not provide information as to whether the isolated and purified DNA segment is being expressed. Expression may be evaluated by specifically identifying the protein products of the introduced isolated and purified DNA segments or evaluating the phenotypic changes brought about by their expression.

Assays for the production and identification of specific proteins and the levels thereof may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an immunoassay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures.

Very frequently the expression of a gene product is determined by evaluating the phenotypic results of its expression. These assays also may take many forms including but not limited to analyzing changes in the chemical composition, morphology, or physiological properties of the plant. Most often changes in response of plants or plant parts to imposed treatments are evaluated under carefully controlled conditions termed bioassays.

### Exemplary Agrobacterium-Mediated Transformation

The following is an example outlining an Agrobacterium-mediated plant transformation. The general process for manipulating genes to be transferred into the genome of plant cells is carried out in two phases. First, all the cloning and DNA modification steps are done in E. coli, and the plasmid containing the gene construct of interest is transferred by conjugation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Also present are restriction endonuclease sites for the addition of one or more transgenes operably linked to appropriate regulatory sequences and directional T-DNA border sequences which, when recognized by the transfer functions of Agrobacterium, delimit the region that will be transferred to the plant.

Plant cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant.

In one possible example, a vector carrying a selectable marker gene (e.g., kanamycin resistance), a cloned Bt toxin fragment coding sequence under the control of the 35S CaMV promoter and the nopaline synthase terminator, is transformed into Agrobacterium. Transformation of leaf tissue with vector-containing Agrobacterium is carried out using standard techniques. Putative transformants are selected after a few weeks on plant tissue culture media containing kanamycin (e.g., 100 μg/ml). Kanamycin-resistant shoots are then placed on plant tissue culture media without hormones for root initiation. Kanamycin-resistant plants are then selected for greenhouse growth. If desired, seeds from self-fertilized transgenic plants can then be used for seed production and, ultimately, for large scale agricultural use. Transfer and expression of transgenes in plant cells is routine practice to those skilled in the art. It has become a major tool to carry out gene expression studies and to obtain improved plant varieties of agricultural or commercial interest. The example given herein are not intended to be limiting, but merely to guide the artisan in understanding the scope of the present invention.

### Pharmaceutical Compositions

As used herein “pharmaceutically acceptable carrier” is intended to include any and all solvents, dispersion media, coatings, antibacterial and antifungal agents, isotonic and absorption delaying agents, anti-inflammatory, stabilizers, and the like, compatible with pharmaceutical administration. The use of such media and agents for pharmaceutically active substances is well known in the art. The indication to be treated, along with the physical, chemical, and biological properties of the drug, dictate the type of formulation and the route of administration to be used, as well as whether local or systemic delivery would be preferred. Except insofar as any conventional media or agent is incompatible with the active compound, use thereof in the compositions is contemplated. Supplementary active compounds can also be incorporated into the compositions. Carrier molecules may be genes, polypeptides, antibodies, liposomes or indeed any other agent provided that the carrier does not itself induce toxicity effects or cause the production of antibodies that are harmful to the individual receiving the pharmaceutical composition. Further examples of known carriers include polysaccharides, polylactic acids, polyglycolic acids and inactive virus particles. Carriers may also include pharmaceutically acceptable salts such as mineral acid salts (for example, hydrochlorides, hydrobromides, phosphates, sulphates) or the salts of organic acids (for example, acetates, propionates, malonates, benzoates). Pharmaceutically acceptable carriers may additionally contain liquids such as water, saline, glycerol, ethanol or auxiliary substances such as wetting or emulsifying agents, pH buffering substances and the like. Carriers may enable the pharmaceutical compositions to be formulated into tablets, pills, dragees, capsules, liquids, gels, syrups, slurries, suspensions to aid intake by the patient. Various formulations and drug delivery systems are available in the art, and a thorough discussion of pharmaceutically acceptable carriers are available in the art (see, e.g., USIP. Remington; The Science and Practice of Pharmacology (Lippincott Williams & Wilkins, 21st ed. 2005); and Ansel & Stoklosa, Pharmaceutical Calculations (Lippincott Williams & Wilkins, 11th ed., 2001)

A pharmaceutical composition used in the methods of the invention is formulated to be compatible with its intended route of administration. Examples of routes of administration include parenteral, e.g., intravenous or intra-arterial, intradermal, subcutaneous, oral or nasal (e.g., inhalation), transdermal (topical), transmucosal, nasal, pulmonary, ocular, gastrointestinal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. Alternate routes of administration include intraperitoneal, intra-articular, intracardiac, intracisternal, intradermal, intralesional, intraocular, intrapleural, intrathecal, intrauterine, intraventricular, and the like.

Pharmaceutical dosage forms of a Cry compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks, or presented in the form of implants and pumps requiring incision. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, emulsions and the like. The compounds may also be formulated in rectal compositions, such as suppositories or retention enemas.

Preferably, pharmaceutical compositions suitable for injectable use include sterile aqueous solutions (where water soluble) or dispersions and sterile powders for the extemporaneous preparation of sterile injectable solutions or dispersion. Depending on the injection site, the vehicle may contain water, synthetic or vegetable oil, and/or organic co-solvents. In certain instances, such as with lyophilized product or a concentrate, the parenteral formulation would be reconstituted or diluted prior to administration. Depot formulations, providing controlled or sustained release of an invention compound, may include injectable suspensions of nano/micro particles or nano/micro or non-micronized crystals. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, poly(ol) (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; dissolution retardant; anti-adherants; cationic exchange resin; wetting agents; antioxidants; preservatives; a disintegrating agent such as alginic acid, Primogel, or corn starch; a lubricant such as magnesium stearate or Sterotes; a glidant such as colloidal silicon dioxide; a preservative; a colorant; a sweetening agent such as sugars such as dextrose, sucrose or saccharin; or a flavoring agent such as peppermint, methyl salicylate, or orange flavoring, each of these being synthetic and/or natural.

For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, emulsion, a solution, a suspension, or a foam, as generally known in the art. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustments; use of complexing agents and other techniques, such as iontophoresis, may be used to regulate skin penetration of the active ingredient.

The active agents can also be prepared in the form of suppositories (e.g., with conventional suppository bases such as cocoa butter and other glycerides) or retention enemas for rectal delivery.

In one embodiment, the agents are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The invention will be further described by the following nonlimiting examples.

### Example I

Cry6A, a 475 residue protein (about 54 kD), is a potent nematicide against many free-living nematode species. In order to determine the minimal essential fragment necessary for activity, over thirty N-terminal and C-terminal truncations were made. Truncated Cry6A DNA sequences were synthesized by PCR using a series of internal primers. BamHI and PstI sites were incorporated into the primers to facilitate fragment recovery. The PCR products were then restriction digested and subcloned into the pQE9 expression vector and transformed into JM103 E. coli.

N-terminal truncations up to, but not including, amino acid residue 11 were effective against C. elegans. Likewise, C-terminal truncations up to, but not including, amino acid residue 382 were also nematicidal. Accordingly, the double truncation resulting in the Cry6A₁₁-382 fragment was toxic to all free-living nematodes tested, e.g., C. elegans and Acrobeloides spp., except P. pacificus. The Cry6A₁₁-382 fragment is estimated to be about 42 kD.

### Example II

### Methods

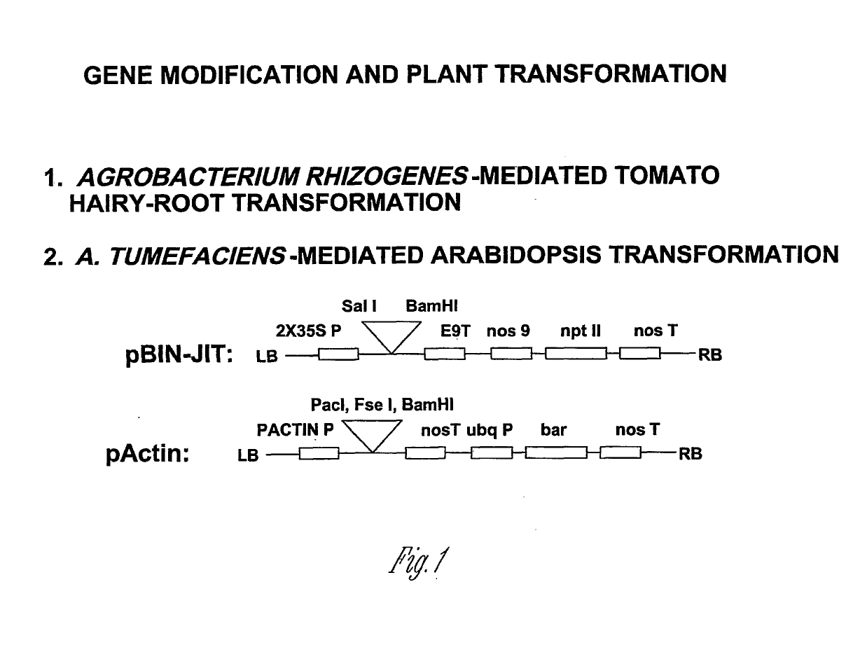

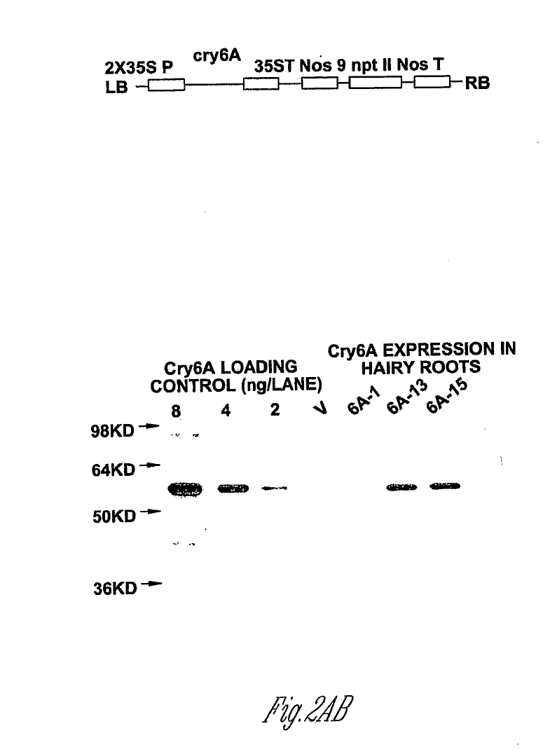

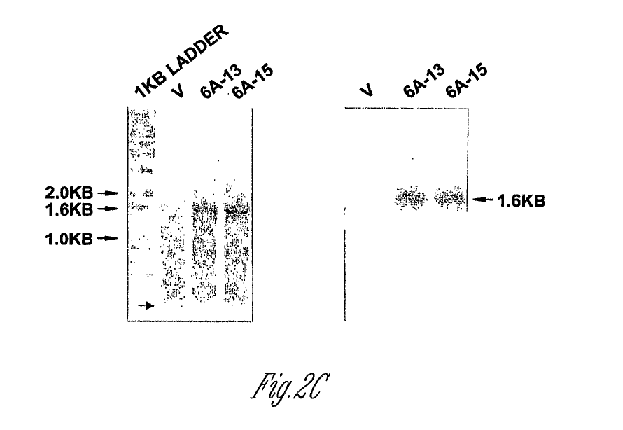

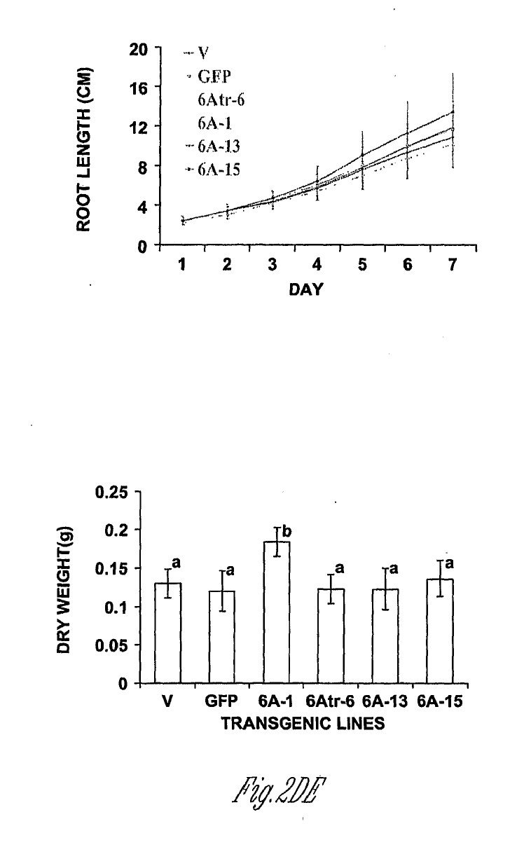

Gene synthesis and vector construction. Plant-version cry6A gene was synthesized by assembling the entire gene de novo from 70-90-mer oligonucleotides with Pfu Turbo DNA Polymerase (Stratagene) and cloning into the pBluescript KS (+) vector. The synthesized cry6A gene was then subcloned into the binary vector pBIN-JIT (Ferrandez, 2000) using SalI and BamHI cloning sites introduced at the 5′ and 3′ ends of the gene and placing cry6A expression under control of the double cauliflower mosaic virus 35S promoter (FIGS. 1 and 2A). Initially, no expression was seen and further modifications were made using the QuickChange multi site-directed mutagenesis kit (Stratagene). These additional changes include adding a plant translation initiation consensus site, which resulted in altering the second amino acid from Ile to Ala, and two prolines at the C-terminus. It was confirmed that bacterially-produced Cry6A with these amino acid alterations is still toxic to C. elegans and Aerobeloides spp. (data not shown). To generate GFP expressing lines, the ³⁵S-GFP vector (Hong et al., 1999) was employed.

Plant transformation. Tomato (Lycopersicon esculentum var. Rutgers select) hairy roots were generated by transforming cotyledons with A. rhizogenes strain R1000, kindly provided by Dr. Nigel Crawford. Briefly, an overnight-grown bacterial culture of R1000 grown in LB medium was centrifuged for 10 minutes at 2,260×g and the pellet resuspended in Murashige and Skoog (MS) medium (Sigma M6899). Tomato seeds were surface sterilized with dilute bleach and germinated in MS medium solidified with 0.8% agar in magenta boxes. After 12 days, cotyledons were removed from tomato seedlings and immersed in the bacterial suspension (OD₆₀₀=0.3) for 30 minutes. These were blotted dry onto a sterilized paper towel, and co-cultivation of the cotyledons and the Agrobacterium was allowed to proceed for 48 hours on MS medium solidified with 2.5 g/L phytagel (Sigma P8169). Following co-cultivation, explants were rinsed with sterile distilled water containing 300 mg/L carbenicillin (Sigma C3416), blotted onto a sterilized paper towel, and then transferred to selection MS medium containing 50 mg/L kanamycin (Sigma K1377) and 300 mg/L carbenicillin. After explants were incubated on a selection medium for 10 days, kanamycin-resistance hairy roots developed. These roots were separated from cotyledons using a sterile scalpel and transferred to fresh MS selection medium once a week for 4 weeks. Only one root line per cotyledon was kept in order to ensure all roots were derived from independent transformation events.

Detection of protein expression in tomato hairy roots. About 100 mg of fresh hairy root tissue was ground in liquid nitrogen to which was then added 200 μl of extraction buffer (100 mM Tris-HCL pH 7.5, 100 mM NaCl, 25 mM DTT, 10 mM EDTA, 5 mM Benzamidine-HCl, 5 mM PMSF, 50 μM Phenanthroline, 1.5 μM Aprotinin, 15 μM Pepstatin A, 20 μM Leupeptin, and 1× proteinase inhibitor cocktail from SIGMA P9599). Following 10 minutes centrifugation at 12,000×g at 4° C., the total soluble protein concentration was determined by Bradford assay (Bio-Rad) using bovine serum albumin (BSA, Sigma A7906) as the protein standard. The protein samples were separated on 10% SDS polyacrylamide gels and immunoblotted onto nitrocellulose membranes. Polyclonal rabbit-anti-Cry6A antibody was used to probe blots containing protein extracts (1 μg/lane) from hairy roots and varying known amounts of full length Cry6A protein expressed in Escherichia coli (from a stock solution whose concentration was determined by comparing intensities of the Cry6A band on Coomassie-stained SDS polyacrylamide gels to known amounts of BSA). Cry6A was detected by the secondary antibody, horseradish peroxidase-conjugated goat anti-rabbit antibody (Jackson ImmunoResearch 111-035-003), and chemiluminescence kit (Super Signal West Pico, PIERCE). GFP expression was confined using an Olympus SZX12 dissection microscope.

RT-PCR and Northern blot analysis. Total RNA was isolated from fresh hairy roots using the RNeasy Plant Mini Kit (Qiagen). For RT-PCR analysis, the first strand of cDNA was synthesized by M-MLV reverse transcriptase (Promega) with oligo d(T) linked to an adapter sequence, 5′-GACGATGAGTCTACCAGGATCCTTTTTTTTTTTTTTTTTTT (SEQ ID NO:4). This cDNA was then PCR amplified using the adapter sequence attached to the oligo d(T) tail (GACGATGAGTCTACCAGGATCC; SEQ ID NO:5) as the 3′ end primer and the very 5′ end of cy6A as the 5′ end primer (CG GTC GAC AAA ATG GCC ATC GAT AGC AAG ACT ACT; SEQ ID NO:6). After amplification, the PCR product was sequenced. For Northern blot, 10 μg of total RNA from each root was electrophoresed in a 1.2% formaldehyde-agarose gel as described in the RNeasy Plant Mini Kit instructions (Qiagen). RNA transfer, probe labeling, hybridization, and washing were carried out according to Sambrook et al. (1989).

Characterization of root growth. For the characterization of main root growth, the main root length was measured for 6 consecutive days after subculture of a 2 cm root tip onto MS medium; each data point represents the average data from 38-60 root tips/line. For the characterization of dry weight, 45-day old hairy root cultures (2 per plate) were dissected out of Gamborg B-5 (Sigma G5893) agar plates. The roots with attached agar were boiled in a microwave to dissolve the agar and then rinsed with hot water. Roots were dried overnight and then in an oven at 90° C. for 5 hours and weighed. The data represent the average from 4 plates/line.

M. incognita maintenance and bioassays. To maintain nematodes on tomato plants, tomato seeds were sterilized with 1:9 dilution in distilled water of 4-6% bleach (Fisher SS290-1) and germinated in MS medium. The resulting seedlings were transplanted to sand that had been washed and autoclaved. After three weeks, the plants were infected with about 25,000 M. incognita eggs. Approximately 8 weeks later, tomato roots were harvested. M. incognita egg masses were hand picked under a dissecting microscope from tomato roots and then sterilized with bleach before infecting new plants.

To set up bioassays of M. incognita infection, eggs were recovered from tomato plants by shaking M. incognita-infected roots in 1:9 dilution of bleach for 3 minutes in a flask. Eggs were collected onto a 25 μm mesh, which were then further bleached twice for 10 minutes with a 1:5:7 dilution of bleach supplemented with 0.02% Tween 20. The eggs were then rinsed four times with sterile ddH₂O. Between each step, eggs were harvested by centrifugation at 500×g for 1 minute. Eggs were hatched at room temperature for 2 days in 100 mg/L carbenicillin, and juvenile 2 stage (J2) worms were collected by allowing them to crawl through 4 layers of Kimwipe papers. These were used for infections. Two 2 cm long roots were transferred to a 150×15 mm plate containing 60 mL Gamborg B-5 medium with 2× Gamborg's vitamins and 0.8% Daishin agar (Brunschwig Chemie, Amsterdam) (all adjusted to pH6.0). After 7 days incubation, root cultures were infected with 50 J2 worms in 400 μL H₂O (25 worms/root). Experiments were carried out at 25° C. in dark. Forty-five days post-infection, the number of galls and egg masses were counted under dissection microscope (it was confirmed with acid fuscin staining that galls are indicative of the presence of nematodes). To count the total number of eggs from each plate, all galls were manually dissected and transferred to 1:9 dilution of bleach in a centrifuge tube. Eggs were suspended with the aid of automatic motor and vortex and then counted under dissection microscope. Three independent sets of bioassays were carried out for all lines, and in any given bioassay a minimum of 5 plates/line were assayed. Thus, each data point represents the average of at least 15 infected plates (30 roots) per line.

Normalization and statistical analysis. By the nature of nematode-plant interactions, we find significant variability from experiment to experiment, as has been noted by others (Urmin et al., 1995). For instance, the average gall number that developed from the vector line from each of the three independent sets of experiment was 24.6, 40.5 and 30.8; the average of egg mass number was 5.9, 16.4 and 10.7; and the average total egg number was 4300, 14742 and 8633. Thus, data from the same set of experiments for all six lines were normalized to the empty vector control line in that given experimental set. In all cases, the same correlation was found in each of the three experiments (e.g., 6A-13 always showed dramatically fewer eggs than vector and GFP-expressing lines). All data were analyzed using ANOVA, and means were compared using Tukey HSD Test at the 5% probability level (http://faculty.vassar.edu/lowry/anovalu.html). Error bars represent the standard error.

Uptake by M. incognita. A total of 40 adult females of M. incognita were individually dissected from Cry6A expressing roots or empty vector-transformed roots under dissection microscope. The dissected worms were rinsed with ddH₂O, transferred to microcentrifuge tube kept on ice, flash-frozen in liquid nitrogen, and then kept in −80° C. Nematodes were sonicated in 25 μL extraction buffer. The samples were then processed for Western blot analyses using affinity purified Cry6A antibody as above.

### Results

Of the four nematicidal Cry proteins that were characterized (Wei et al., 2003), Cry6A was selected for initial testing against PPNs because: 1) Cry6A is toxic to the free-living nematode Acrobeloides spp., which is phylogenetically closely related to PPNs, and 2) it is the smallest of the nematicidal proteins, making it the easiest to synthesize. Bacterial genes encoding Cry proteins need to be resynthesized with altered codons since bacterial genes have different codon preferences, and contain sequences interpreted by plant cells as polyadenylation sites, introns, or mRNA destabilization factors (DeRocher et al., 1998; Diehn et al., 1998). A cry6A gene was prepared that avoids rare plant codons and sequences that resemble polyadenylation sites and introns. Since this first version of cry6A was not expressed in hairy roots, additional codons were altered that might remotely resemble problematical sequences: a plant translation initiation sequence (AAAATGGC) was introduced at the 5′ end, and 2 extra proline codons were introduced at the 3′ end of cry6A to protect the C-terminus from proteases. Approximately, 25% of the nucleotides were altered relative to the original Cry6A gene, and the G-C content increased from 27% to 44% (SEQ ID NO:1).

The codon-modified cry6A gene was cloned downstream of the double cauliflower mosaic virus (CaMV) 35S promoters (FIG. 2) and introduced via Agrobacterium rhizogenes into tomato cotyledons to generate transgenic hairy roots. This hairy root system is a simple and relatively rapid means to express proteins in plant roots and has been used to study the effects of transgene expression in roots on various nematodes, including M. incognita (Hwang et al., 2000 Plovie et al., 2003), Globodera pallida (Urwin et al., 1995), and Heterodera schachtii (Cai et al., 1991).

Transgenic roots expressing both full-length Cry6A protein (54 kDa) (18 lines) and a truncated Cry6A protein (43 kDa; nucleotides 1-1158) (15 lines) were generated. Lines transformed with a truncated Cry6A construct also were generated as a means to test dose-dependency since truncated Cry6A is less toxic to C. elegans than full-length Cry6A (Wei et al., 2003). Amongst the independent hairy root lines generated, two (6A-13 and 6A-15) expressed full length Cry6A at 0.25% total soluble protein (FIG. 2B) and one (6Atr-6) expressed truncated Cry6A at a similar level (data not shown). These three are the highest expressing lines generated. Both sequencing of RT-PCR products and Northern blot analysis (FIG. 2C) confirmed that cry6A transcripts in transgenic roots are complete and correct at the nucleotide level. Three control hairy root lines were generated that were transformed with: 1) empty-vector (V), 2) green-fluorescent protein (GFP) gene under CaMV35S promoter, and 3) full length cry6A gene under CaMV35S promoter but that failed to express any protein (6A-1). All six root lines had similar growth rates, with the exception of 6A-15, which grew faster (FIG. 2D). All lines also displayed similar 45-day total root dry weights, with the exception of 6A-1 that had slightly thicker roots and more branches (and thus weighed slightly more). In general, the roots of the different lines are morphologically well matched; there are no major differences amongst them. Of particular note, the GFP and 6A-13 lines are nearly identical in measured and visible characteristics.

To test whether M. incognita is intoxicated by the Cry protein, control and Cry6A-expressing lines were inoculated with freshly hatched M. incognita juveniles. After 45 days, the frequency of M. incognita infection and production of progeny were then compared. Only slight differences in the number of galls produced were detected, with Cry6A lines showing a small reduction in gall numbers compared to control lines (FIG. 2F). However, the smaller root knots seen in Cry6A-expressing lines suggested that Cry6A was adversely affecting the health of the nematodes.

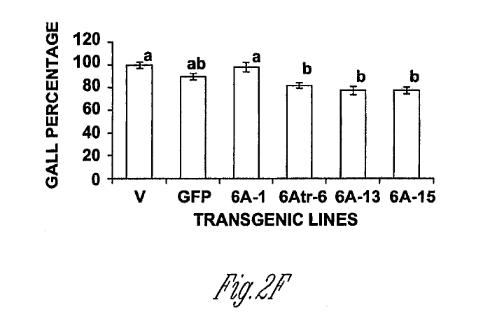

Next, it was tested to see whether the crystal toxin was affecting the reproduction of M. incognita. Nematode reproduction is very sensitive to the effects of Cry protein intoxication (Wei et al., 2003). For example, strong inhibition of progeny production can occur in free-living nematodes at Cry protein concentrations that are >10-fold lower than the LC₅₀. It was found that in the two lines expressing Cry6A, 6A-13 and 6A-15, the number of egg masses respectively decreased to 62% and 50% relative to that of the empty vector line (FIG. 3B). Even more dramatic differences are seen in the production of total progeny by the parasites. The number of eggs produced from the Cry6A-expressing lines 6A-13 and -15 were reduced to 36 and 24% of progeny production in vector-only control and was significantly lower than that of all three control lines. Eggs produced from 6Atr-6 were reduced to 44%, consistent with the fact that truncated Cry6A is toxic to nematodes, but at a reduced level (Wei et al., 2003). This result is consistent with a dose-dependent effect of Cry6A on M. incognita.

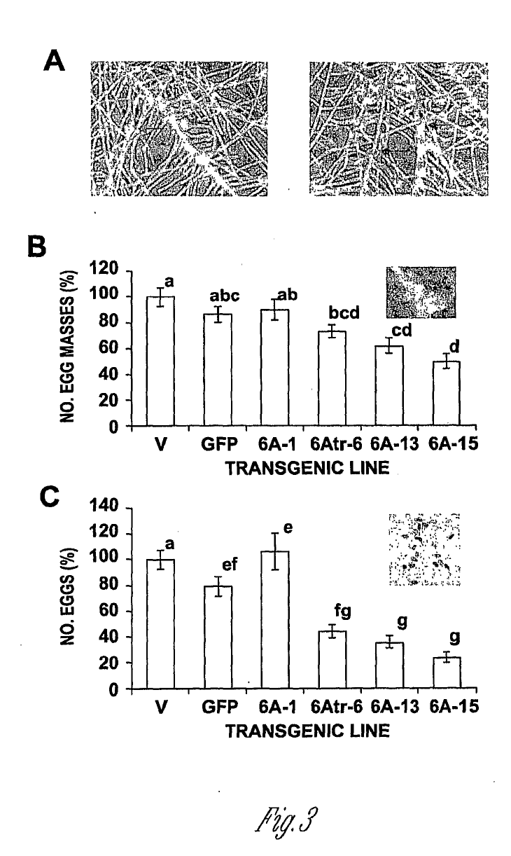

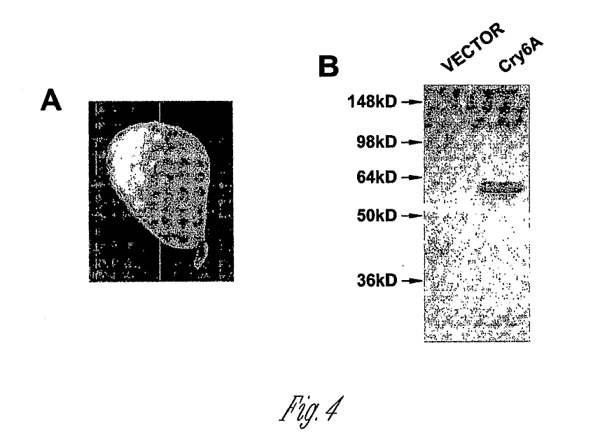

The physiological mechanism of action of Cry proteins requires that the proteins be ingested (deMaagd et al., 2001). To confirm that M. incognita is ingesting Cry6A, M. incognita females were dissected out of both Cry6A expressing and vector control lines (FIG. 4A). Proteins were extracted from both sets of nematodes, separated by SDS PAGE, and then subjected to Western blot analysis in order to detect Cry6A protein. Full length Cry6A protein was detected from nematodes feeding on Cry6A expression lines but not nematodes feeding on vector-only transformed lines (FIG. 4B). These data, and the fact that Cry6A is able to intoxicate the nematode, indicate that M. incognita is ingesting the 541 kDa Cry6A protein.

These results demonstrate for the first time that Cry proteins can intoxicate PPNs. Ingestion of Cry6A by M. incognita resulted in up to a 4-fold reduction in the amount of progeny produced by females infecting tomato roots expressing Cry6A. The efficacy of Cry6A intoxication may be enhanced in the future by increasing Cry6A expression level and/or by engineering the Cry6A protein to have greater toxicity via alteration in its primary sequence, as has been done for other Cry proteins (Wei et al., 2000).

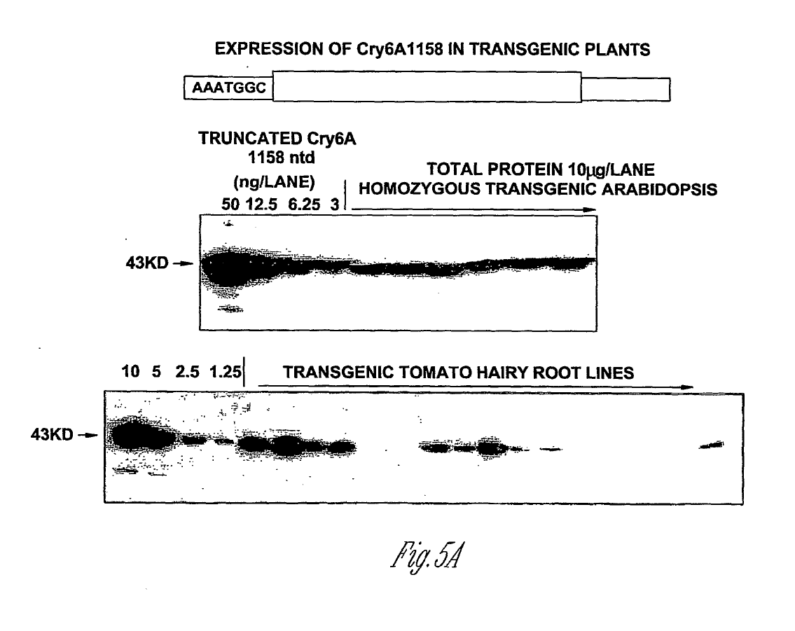

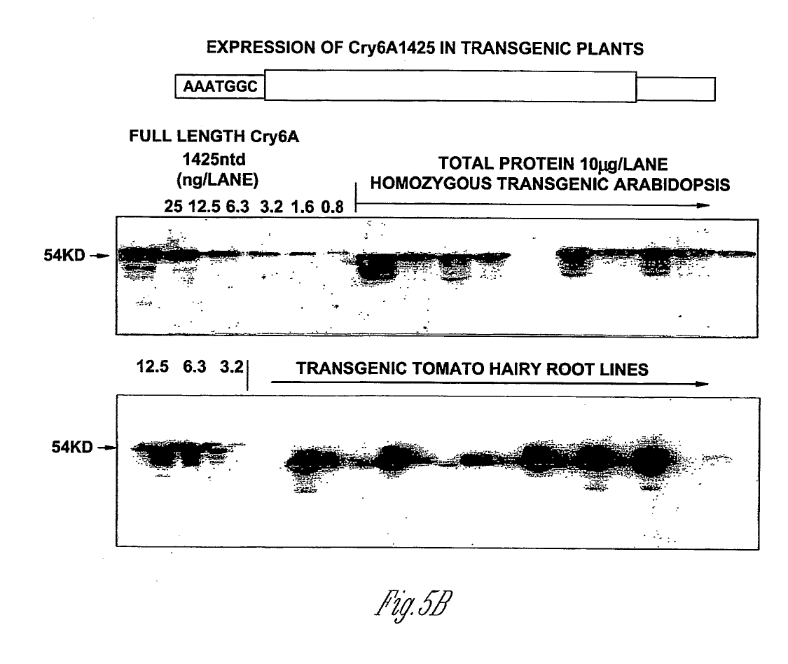

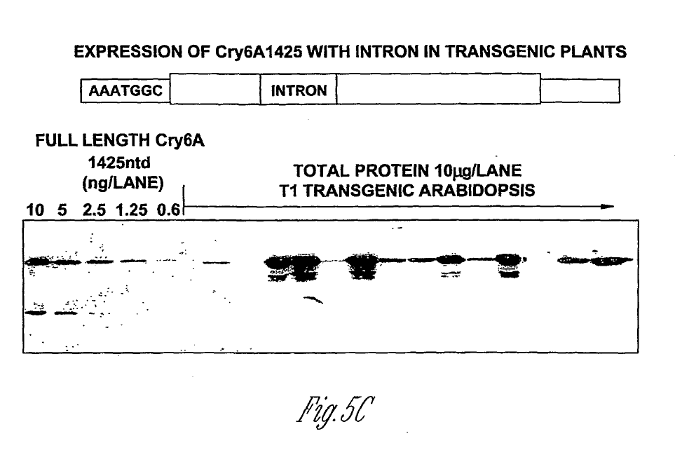

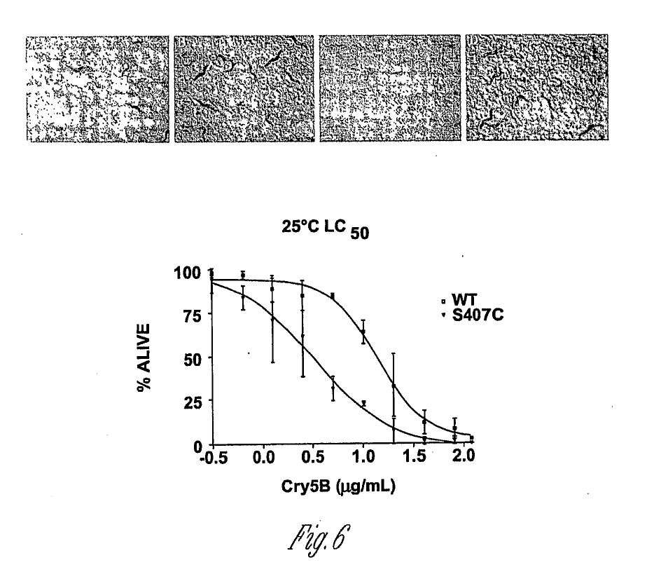

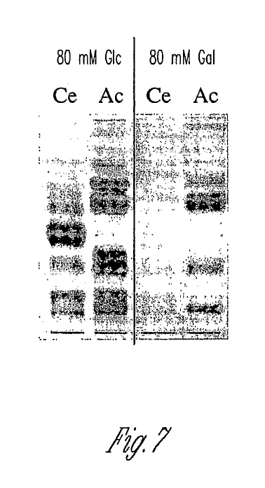

Expression data for transgenic Arabidopsis and tomato hairy root lines is shown in FIG. 5.

These results also demonstrate for the first time that a sedentary PPN can ingest a protein larger than 30 kDa. Indeed, one of the key reasons why Bt Cry protein as potential nematicidal agent perhaps has been overlooked is because the beet cyst nematode, Heterodera schachtii, is unable to ingest proteins larger than about 30 kDa (Bockenholt et al., 1994; Urwin et al., 1997). However, similar experiments failed to determine the same −30 kDa maximum limit for three other PPNs, including M. incognita (Urwin et al., 1997), Globodera vostochiensis (Goverse et al., 1998), and Rotylenchulus reniformis (Urwin et al., 1998). These results indicate that larger proteins, including Cry proteins, are now viable candidates for biocontrol of PPNs and thus open up a whole new range of options for control of nematodes, with the possible exception of Heterodera.

The four-fold reduction in progeny production brought about by Cry6A expression in plants has potentially great utility, especially as part of an integrated pest management system. Cry6A could be “stacked” or combined with non-transgenic crop varieties, e.g., tomato, cotton, and potato, with nematode-resistant traits (Williamson et al., 1996), providing a major boost to nematode control in these varieties. Since Cry6A suppresses nematode reproduction, it would also delay the evolution of nematode resistance to these crop traits. Stacking of Cry6A with other, mechanistically different biocontrol options, such as proteinase inhibitors (Urwin et al., 1995; Urwin et al., 1998; Atkinson et al., 2004) or double-stranded RNA (Urwin et al., 2002), would offer similar advantages. It is predicted to be difficult for nematodes to resist toxicity associated with such stacked plants. Cry6A expression could also provide a viable alternative to crop rotation strategies. Given the results presented here and the long and distinguished track record of Bt Cry proteins for insect control, expression of crystal proteins in plants can be a potentially useful strategy for biocontrol of a wide range of plant parasitic nematodes.

In summary, M. incognita was able to ingest Cry6A expressed in transgenic roots and Cry6A significantly impaired the ability of M. incognita to reproduce. These results establish for the first time that a Cry protein can achieve biocontrol of PPNs.

### Example III

### Exemplary Mutagenesis Protocol

Cry5B is mutagenized (about 1-3 amino acid substitutions per gene) using a mutagenesis kit (Strategene). These mutated Cry5B clones, in an IPTG-inducible vector, are transformed into E. coli, and colonies are hand picked into a 96-well microtiter plate and grown overnight. Aliquots are pipetted onto four 24-well NG agar plates with IPTG, inducing expression of the clones. Mutant clones that express a hypertoxic Cry5B can be detected by looking for wells with worms that are sicker than control wells that contain non-mutated Cry5B after 6 days at 25° C.

1920 clones of random-mutagenized Cry5B clones were screened for those with increased toxicity. Twelve candidates were reconfirmed as hypertoxic based on the isolation of the plasmid with the mutated Cry5B, retransformation into a new E. coli strain (JM103), and retesting against wild-type Cry5B in JM103. SDS PAGE was carried out for 12 clones, and it was confirmed that all expressed normal (not elevated) levels of Cry5B. Four of twelve hyperactive candidate clones have been identified bearing various point mutations.

Two of the four Cry5B variants were mutation-corrected to determine which point mutations resulted in increased toxicities. That is, re-mutagenesis of single amino acids in each of these clones back to the wild-type sequence reverted the clone back to the same toxicity as wild-type Cry5B. One such mutation is Serine 407 to Cystine (S407C) and another is Serine 663 to Proline.

### Results

Cry6A may be truncated up to nucleotide 1146 (i.e., retains nucleotides 1-1146; full length is 1425 ucleotides), and is still toxic to C. elegans (Example I). Cry6A can be truncated up to nucleotide 1158 and still retains toxicity against the plant parasitic nematode M. incognita (a major crop pest) when the protein is expressed in transgenic tomato roots (Example II). To improve the toxicity of Cry6A truncated at nucleotide 1158 against C. elegans, amino acid substitutions were introduced. This was done using random mutagenesis and Cry6A/Cry6B swapping approach. The following clones with truncated mutant Cry6A genes were found to be potentially more toxic against C. elegans:

| | |
|---|---|
| E8 | K19N, K192E, K205R, K249R, Q319L |
| E35 | L138V |
| E44 | Q52L, T235A |
| E68 | K170N |
| E90 | K99N |
| ES7 | G121S |
| ES24 | Q62R, D147G |
| ES34 | I164T, E237G |

C. elegans was inoculated on wild-type Cry6A(1-1158) for one month. C. elegans inoculated for one month on the E44 mutant Cry6A(1-1158) was unable to grow. Moreover, expression of a Cry6A gene with a single substitution found in E44 (Q52L), had increased activity.

A series of Cry5B truncations were made and tested against C. elegans. Full length Cry5B has 3735 nucleotides (1245 amino acids). Truncations at nucleotide 2142 (714 amino acids) and at nucleotide 2094 (698 amino acids) were still toxic against C. elegans. Any truncation less than or equal to 933 amino acids lay within a >25% limit.

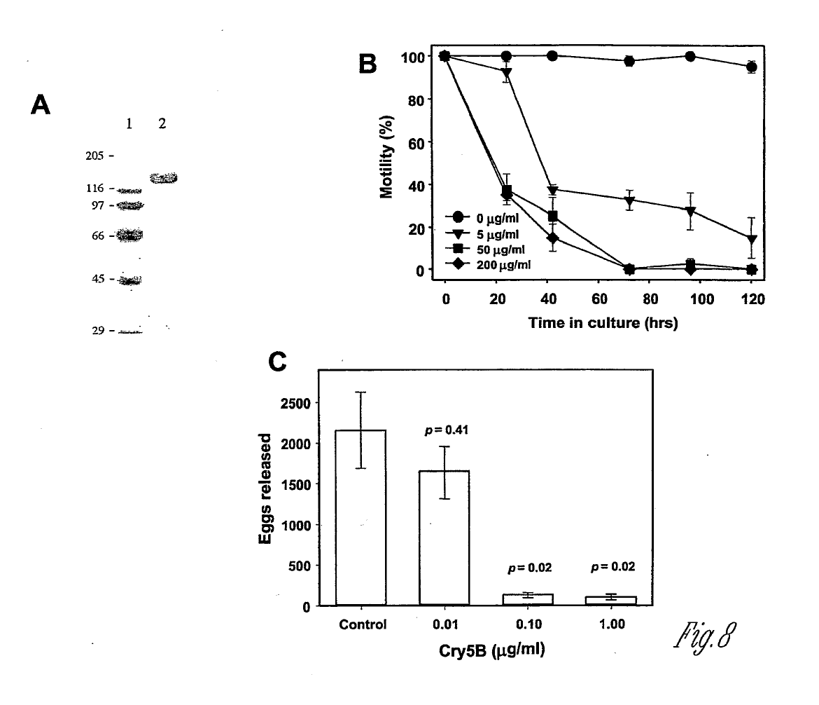

A similar mutation approach was employed with Cry5B as with Cry6A and it was found that S407C and S663P substitutions resulted in increased activity against C. elegans. To quantitate how hypertoxic the S407C mutation was relative to wild-type Cry5B, the mutant Cry5B was subcloned into a Bt expression vector and expression in Bacillus thuiengiensis. Cry5B(S407C) and Cry5B(Wt) were purified using standard protocols and used in LC₅₀ assays. The results indicated that S407C is 3.7 fold more toxic than wild-type Cry5B at 25° C. (P=0.029 paired t-test; FIG. 6).

Cry5B and various proteolytically processed variants were used in a quantitative binding assay to immobilize worm glycolipids. Full length Cry5B (FL) and elastase-treated Cry5B (elastase) both bound very well to glycolipids. Acid extract protease treated Cry5B (AE pH 3.0) did not bind well. Elastase cleaves the protein at about amino acid 769, and acid extract cleaves the protein at about amino acid 718. These data suggest that binding for Cry5B to its glycolipid receptor resides between amino acids 718 and 769. Furthermore, Cry5B processed to amino acid 769 is as effective in binding receptor as well as the full length toxin. These data suggest that truncation of the protein at or near amino acid 769 should be sufficient to produce a potent nematicidal agent.

Griffitts et al. (2005) showed that the binding receptors for Cry5B are nematode-specific glycolipids. Since parasitic worms shed a lot of carbohydrate into human hosts, Cry5B and/or the glycolipid-binding fragment of Cry5B may be employed as a sensitive diagnostic tool for parasitic nematode infections in humans and other mammals and vertebrates.

### Example IV

### Materials and Methods

Parasites and Hosts. The Ancylostoma ceylanicum life cycle was maintained in 3-4 week-old male golden Syrian hamsters of the HsdHan:AURA outbred strain as described in Bungiro et al. (2001) and Garside et al. (1989). For the in vivo anthelminthic study, hamsters were orally infected with 150 third-stage A. ceylanicum larvae, treated as described below, and sacrificed at day 22. The maintenance and care of experimental animals complied with the National Institutes of Health guidelines for the humane use of laboratory animals and were approved by the Yale University Animal Care and Use Committee.

Expression and purification of Cry5B protein. Cry5B was purified from a crystal-toxin less Bt strain HD1 transformed with a plasmid containing the Cry5B gene under control of its own promoter and containing a gene for erythromycin resistance (Marroquin et al., 2000). Four milliliters of a saturated overnight culture of Cry5B-Bt was inoculated into 200 mL PGSM media (Brownbridge et al., 1986) containing 10 μg erythromycin/mL, and then allowed to sporulate for 5 days at 30° C. with shaking (250 RPM). All subsequent steps were carried out at 4° C. unless indicated. The crude spore-crystal lysate (SCL) was centrifuged at 7400×g (7000 rpm in a Sorvall SLA-1500 rotor) for 7 minutes and then suspended in 5 mL 1.0 M NaCl. The SCL was sonicated twice for 1 minute intervals, an additional 25 mL 1.0 M NaCl was added, and then centrifuged as before. The SCL was washed twice more in 30 mL volumes of 1 M NaCl, twice in 30 mL ddH₂O, and then suspended in 8 mL ddH₂O. Cry5B crystals were purified using a sucrose gradient (Debro et al., 1986) with some modifications. Briefly, four mL of the lysate was layered on a step gradient of 8.3 mL 60% sucrose, 5.0 mL 40% sucrose, 8.3 mL 30% sucrose and 8.3 mL 10% sucrose. The gradient was centrifuged at 4500×g in an SW-28 rotor (Beckman) for 20 minutes. The Cry5B crystal band was removed and diluted 5 fold with water. The pellet from the sucrose spin was suspended in ddH₂O and centrifuged on a second sucrose gradient as before. The Cry5B band from this spin was also diluted 5 fold in water. The Cry5B crystals were washed 3 times in ddH₂O, with centrifugation at 25,000×g (Sorvall SS-34 rotor) over 30 minutes for the first wash and 15 minutes for the next two washes. Crystals were suspended in 1 mL ddH₂O, and Cry5B was solubilized by addition of 30 mL 50 mM potassium citrate (pH 3.0), 10 mM dithiothreitol. Solutions were gently rocked for 2 hours at room temperature and then centrifuged at 12,000×g (Sorvall SS-34 rotor) for 10 minutes. The supernatant was filtered through a 0.22 μM syringe filter (Millipore). Cry5B was precipitated by addition of 877 μL 1 M tripotassium citrate and incubated on ice overnight. Cry5B was centrifuged at 3,500×g for 5 minutes and suspended in water as a precipitate. Aliquots were removed and solubilized in 20 mM Hepes (pH 8.0) to determine protein concentration using a Bradford assay (Bio-Rad). Cry5B was frozen in aliquots in liquid N₂ and stored at −80° C. On the day of use Cry5B aliquots were thawed, centrifuged at 15,000×g for 5 minutes, and the supernatant removed. The precipitated Cry5B was then resuspended to a final concentration of 5 mg/ml in 50 mM potassium citrate buffer, pH 3.0, immediately prior to gavage.

In vitro assay of Cry5B toxin binding activity. Upper phase glycolipids from C. elegans and A. ceylanicum were extracted as described in Griffitts et al. (2005), except that A. ceylanicum adults were partially homogenized with a plastic pestil and extracted overnight. Equal amounts by mass (about 500 ng based on orcinol staining) of upper phase glycolipids from C. elegans and A. ceylanicum were separated by thin layer chromatography. Overlay experiments using biotinylated Cry5B were carried out as described in Griffitts et al. (2005), either in the presence of 80 mM glucose or 80 mM galactose.

In vitro assays of Cry5B toxicity against hookworm life cycle stages. In order to obtain adult hookworms for ex vivo study, hamsters were infected orally with 150-200 third-stage A. ceylanicum larvae. At 20 days post-infection (PI), the animals were euthanized and intestines dissected longitudinally. Live parasites were gently removed from the intestinal debris with fine-tipped forceps and cultured as described below. For studies of the effect of toxin on A. ceylanicum egg release, groups of 3 adult female hookworms were placed in wells of a microtiter plate containing RPMI (Invitrogen)/50% fetal calf serum (FCS) (Sigma) with 2× Penicillin-Streptomicin (Pen-Strep) (Invitrogen) and fungizone (10 μg/ml) (Invitrogen). After 24 hours incubation at 37° C., the adult worms were removed and the eggs suspended in 500 μL of RPMI. The total number of eggs in the suspension was extrapolated from mean values obtained by counting the eggs in 6 separate aliquots (10 μL) from each well. Each treatment group consisted of three replicate wells. For studies of egg hatching and early larval toxicity, aliquots of eggs released from adult females over 24 hours in the absence of toxin were placed in individual wells of a microtiter plate containing RPMI/50% FCS/2× Pen-Strep/Fungizone and increasing concentrations of Cry5B. The number of larvae in each well was counted at 24 hour intervals using light microscopy. In addition, the motility of hatched larvae in each treatment group was determined during a 30 second period of observation under light microscopy. For adult worms, the effect of Cry5B oil motility was measured by incubating groups of 10 worms (5 males, 5 females) in wells of a 24 well microtiter plate containing 1 mL of RPMI/50% FCS/2× Pen-Strep/Fungizone and increasing concentrations of toxin. As above, motility was determined at various times over 120 hours by observation under light microscopy. Photomicrographs of worms were obtained using a microscope equipped with a digital camera. For all in vitro experiments, Cry5B was solubilized in citrate buffer (see above) prior to adding to wells.

Effect of anthelminthics on hookworm infection. Weanling hamsters (n=30) were orally infected with 150 A. ceylanicum or left uninfected to serve as age-matched controls (n=10). On days 14, 15, and 16 PI, infected hamsters (n=10 each group) were given 1 mg Cry5B in potassium citrate buffer or 1 mg mebendazole (Sigma) in distilled H₂O, each delivered orally in a volume of 0.2 ml. Infected control animals (n=10) received 0.2 ml citrate buffer only. Hamsters were monitored for weight, hemoglobin, and (when applicable) fecal eggs. Blood hemoglobin was measured using the Total Hemoglobin assay kit (Sigma Diagnostics, St. Louis, Mo.). Sample values were determined using a hemoglobin standard curve prepared from reagents provided in the kit. Fecal egg counts were performed using a McMaster chamber (Hausser Scientific, Horsham, Pa.). At day 22 infected hamsters were sacrificed and adult hookworms removed manually from the small and large intestines.

Statistical Analysis of Data. Data are presented in the text and figures as means +/− standard error. Significance testing was conducted using the StatView 4.51 statistical analysis software package (Abacus Concepts, Inc., Berkeley Calif.). For multiple group comparisons analysis of valiance (ANOVA) was perfomed followed by Fisher's Protected Least Significant Difference as a post test. P values of <0.05 were considered significant.

### Results

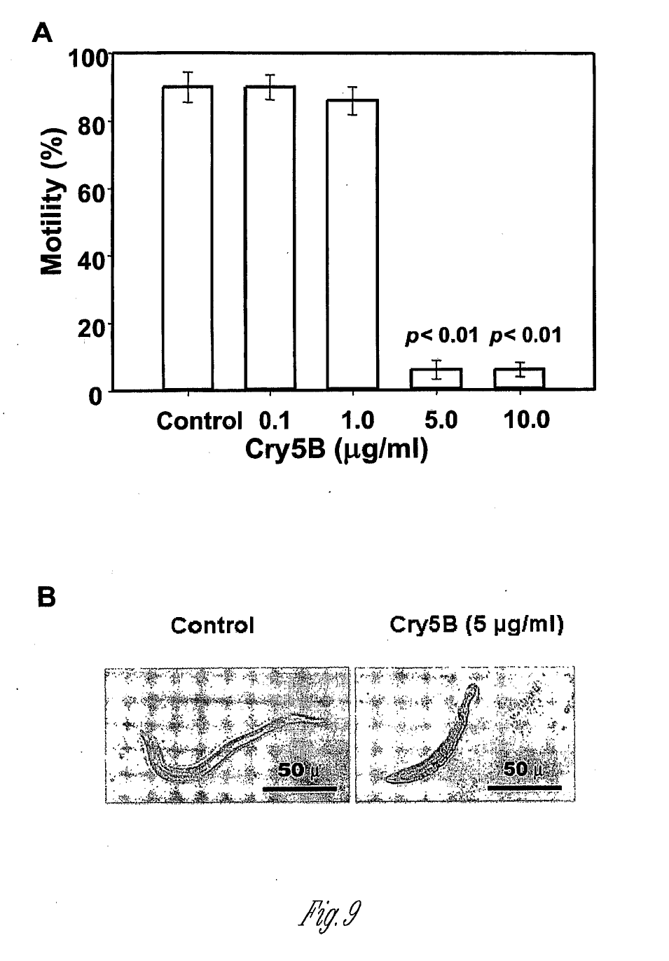

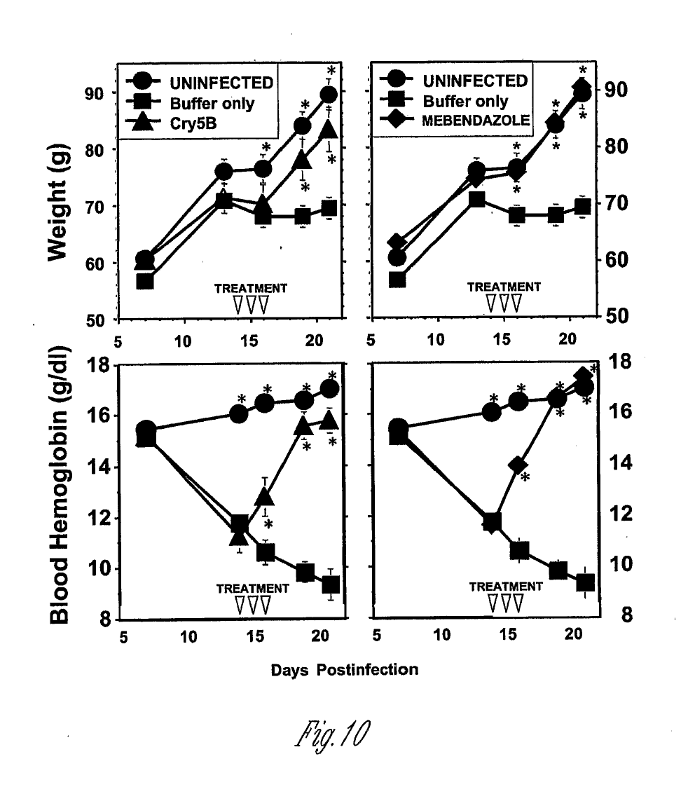

Adult hookworms bind Cry5B toxin in vitro. In order to confirm that the hookworm A. ceylanicum was potentially susceptible to the toxic effects of the nematicidal Cry protein, Cry5B, it was first investigated whether A. ceylanicum adults express a Cry5B receptor. As previously shown, the receptor for Cry5B in C. elegans are invertebrate-specific glycolipids, specifically the carbohydrate moieties present on these glycolipids (Griffitts et al., 2005). Since glycolipids are well conserved among nematodes (Lochnit et al., 2000), it was hypothesized that Ancylostoma hookworms would similarly express Cry5B receptors. Glycolipids were extracted from C. elegans and A. ceylanicum, resolved by thin layer chromatography (TLC), and an overlay/binding experiment performed using activated, biotinylated Cry5B protein. Cry5B binds multiple glycolipid species in both C. elegans and A. ceylanicum. Binding of Cry5B to C. elegans glycolipids was specifically inhibited in the presence of galactose, but not glucose. Hookworm receptors demonstrated similar specificity, since addition of 80 mM galactose to the binding experiment reduced (but did not eliminate) binding of Cry5B to glycolipids from A. ceylanicum (FIG. 7). These data confirm that A. ceylanicum expresses Cry5B glycolipid receptors.

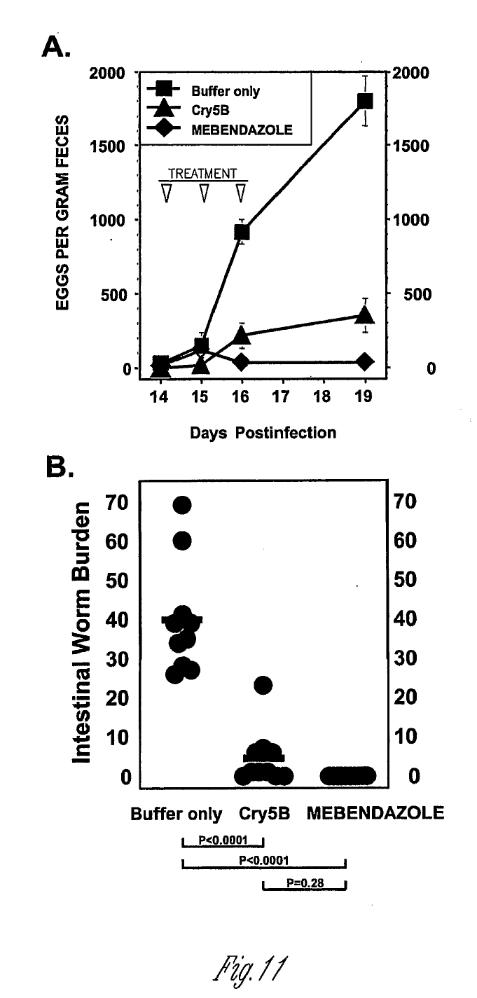

Effect of Cry5B toxin on adult hookworms in vitro. The susceptibility of A. ceylanicum to a nematicidal Bt toxin was evaluated by culturing adult worms in the presence of increasing concentrations of purified recombinant Cry5B. Before using Cry5B in these assays, additional steps were added to previous Cry5B purification procedure (Griffitts et al., 2001) in order to achieve greater levels of Cry5B purification. As shown in FIG. 8A, the toxicity of Cry5B against adult worms was noted at all concentrations tested. By 24 hours incubation, the motility of adult worms was already significantly reduced in the presence of 50 μg/mL and 200 μg/mL Cry5B. By 42 hours incubation, statistically significant reductions in motility were seen at all toxin concentrations (5-200 μg/mL), and by 72 hours there was complete loss of motility in both the 50 μg/ml and 200 μg/mL Cry5B groups. In contrast, the motility of adult hookworms maintained in standard culture media (without toxin) remained at 95% or greater throughout the course of the observation period. Exposure of adult worms to Cry5B also resulted in significant morphologic changes consistent with severe toxicity (not shown). Treated worms demonstrated a marked distortion or ruffling of the cuticle compared to controls. This effect was seen at all concentrations of toxin tested (5-200 μg/mL), but was most pronounced in worms exposed to higher concentrations of Cry5B.

Incubation in the presence of Cry5B toxin also reduced egg release by adult female hookworms in a concentration dependent manner (FIG. 8B). Groups of adult female A. ceylanicum were exposed to a range of toxin concentrations (0.01-1 μg/mL) for 24 hours, at which time the number of eggs released was determined. Control worms cultured in the absence of toxin released a mean of 2150+/−475 eggs. Worms cultured in the lowest concentration of toxin (0.01 μg/ml) excreted fewer eggs (1634+/−320), although this difference was not statistically significant. In contrast, females exposed to 0.1 μg/ml and 1 μg/ml Cry5B released about 1/20^{th} the number of eggs (respectively 121+/−31 and 97±33) as control females, reductions that were both statistically significant. These concentrations of toxin did not impair motility of the adult females over the period of observation and are consistent with previous observations in free-living nematodes that progeny production is particularly sensitive to the intoxicating effects of nematicidal Cry proteins (Wei et al., 2003).

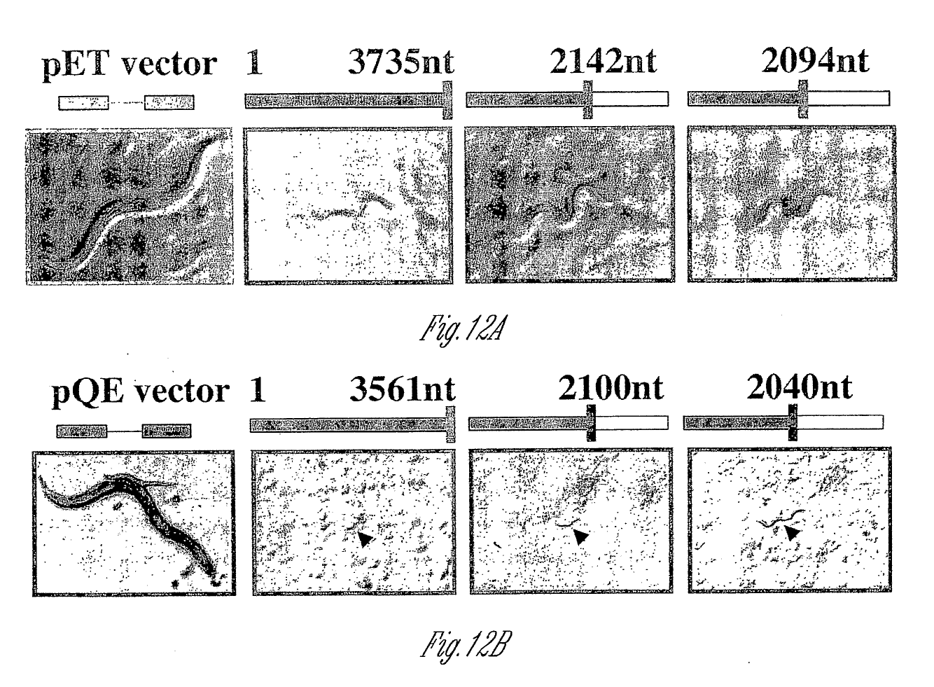

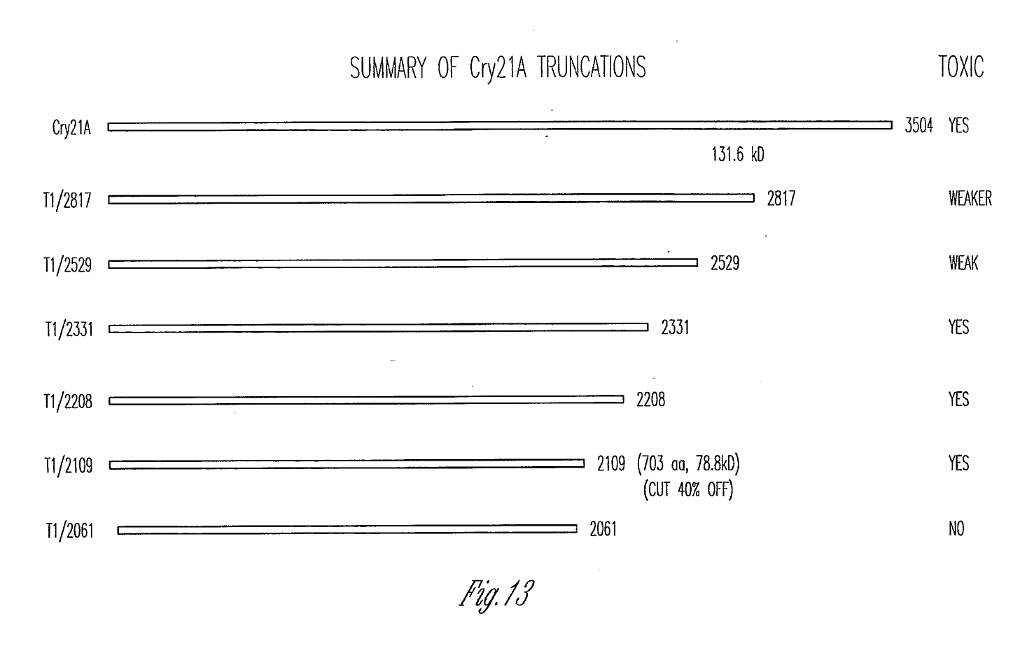

Effect of Cry5B toxin on hookworm larval development in vitro. The susceptibility of early developmental stages of A. ceylanicum to Cry5B was evaluated by incubating eggs harvested from adult female hookworms in the presence of increasing concentrations of purified recombinant toxin. The number and motility of L1 larvae released from these eggs was monitored daily and compared to worms maintained in standard media (RPMI/50% FCS). Purified Cry5B toxin had no effect on egg hatching, with comparable numbers of larvae released over 48 hours in all treatment groups (range 29+/−1 to 36+/−4 per well) consistent with the fact that Cry proteins are ingested toxins and that embryos do not feed. However, there was a concentration dependent effect of Cry5B on the motility of larvae released from eggs at 48 hours. In the absence of Cry5B, 90+/−4% of cultured larvae demonstrated motility at 48 hours observation (FIG. 9A). There was no significant difference in the motility of larvae hatched in the presence of 0.1 μg/mL (90+/−3%) or 1.0 μg/ml (86+/−4%) Cry5B (p>0.6 for each group compared to control). At a concentration of 5 μg/mL Cry5B, however, larval motility was reduced to 6+/−3%, a difference that was statistically significant. A similar effect was seen in larvae cultured in the presence of 10 μg/mL Cry5B, with 6+/−2% motility (p<0.01 compared to control) observed at 48 hours. The data thus confirm that early larvae are more sensitive to the effects of Cry5B than adult hookworms, consistent with results from studies on free-living nematodes (Wei et al., 2003). These data also suggest that there may be a threshold concentration above which newly hatched A. ceylanicum larvae are susceptible to the effects of Cry5B.

Incubation with Cry5B also resulted in significant morphological changes to A. ceylanicum larvae, as demonstrated by light microscopy (FIG. 9B). Hookworm larvae exposed to toxin exhibited stunted growth and showed obvious loss of integrity of most internal structures. These findings confirm that early larval stages of A. ceylanicum are highly susceptible to intoxication with Cry5B.

The susceptibility of A. ceylanicum L3, which is the infectious stage of hookworm, to Cry5B was evaluated by incubating larvae cultured from the feces of infected hamsters in the presence of increasing concentrations of toxin. Worms were monitored daily for evidence of intoxication by determining motility under light microscopy. These non-feeding third stage larvae, which possess a dense external sheath that covers the buccal capsule, were resistant to the effects of Cry5B up to concentrations of 10 mg/mL (data not shown), consistent with the fact that Cry proteins need to be ingested to be active.

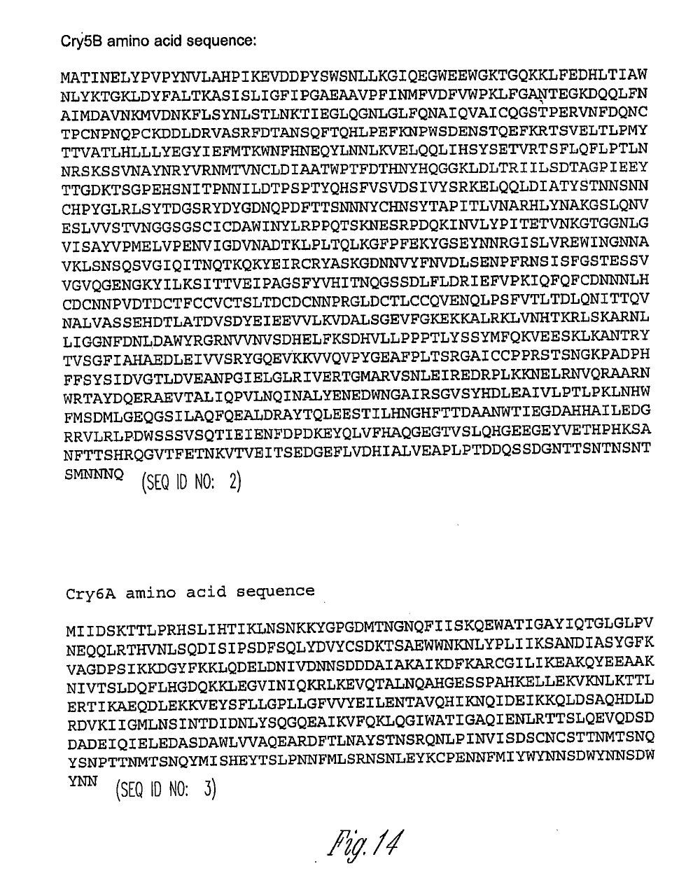

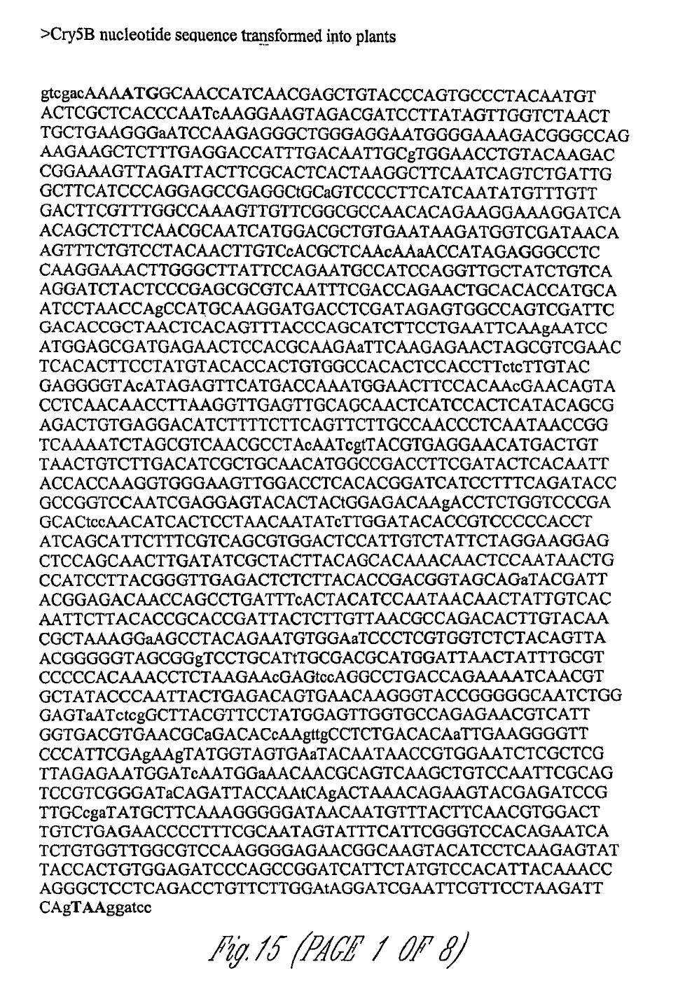

Cry5B Treatment Reduces Pathology in Hookworm Infected Hamsters. Previously, it was demonstrated that hamsters infected with A. ceylanicum exhibit profound weight loss and anemia (Bungiro et al., 2001; Held et al., 2000; Chu et al., 2004; Bungiro et al., 2004). In order to evaluate the therapeutic potential of Cry5B, hamsters (n=30) were infected with 150 A. ceylanicum larvae and groups of 10 animals were orally dosed on days 14, 15, and 16 post-infection with buffer only, Cry5B, or the benzimidazole anthelminthic mebendazole (the standard treatment for hookworm infection). As expected, relative to uninfected animals, the infected hamsters in the buffer-treated group exhibited weight loss and significant reductions in blood hemoglobin levels temporally associated with the onset of hookworm bloodfeeding, which occurs at approximately day 10 post-infection (PI) (FIG. 10). Persistent and statistically significant differences in weight were observed between the buffer-treated and uninfected control animals beginning at day 14 PI, while a difference in blood hemoglobin level was detected by day 16 PI. In contrast, treatment with Cry5B was associated with significantly improved weight levels in infected animals (FIG. 9A, top left panel). By day 19 PI the difference in weight between the Cry5B-treated animals (mean 77.9+/−3.6 g) and buffer-treated controls (mean 67.9+/−1.9 g) was highly statistically significant (p=0.008). This difference was even more pronounced by day 21 PI, with a mean weight of 83.2+/−3.9 g in the Cry5B treated group vs 69.2+/−1.9 g in the buffer-treated animals (p=0.0008). Of note, by day 19 PI the mean weights of the Cry5B-treated animals were found to be statistically equivalent to those of the uninfected hamsters (p 0.1), as well as to those of infected hamsters treated with mebendazole (p=0.08) (FIG. 10 upper right panel).

Cry5B treatment was also associated with rapid resolution of hookworm anemia, as measured by blood hemoglobin levels (FIG. 10B bottom left panels). Prior to treatment (day 14 PI), the mean blood hemoglobin levels in each of the three infected groups (buffer, Cry5B, mebendazole) ranged from 11.2 to 11.7 g/dL, compared to a mean of 16.0+/−0.2 g/dL in the uninfected control animals (p<0.001 for each infected group vs uninfected controls). However, a significant improvement in blood hemoglobin levels was noted in the Cry5B treatment group (mean 12.8+/−0.8 g/dL) compared to the buffer-treated controls (mean 10.6+/−0.5 g/dL) by day 16 PI (p=0.003), which was the third treatment day. The improvement in blood hemoglobin was sustained throughout the study period (p<0.0001 vs buffer-treated animals at days 19, 21). As was observed for weight, the post-treatment blood hemoglobin levels (Day 19, 21) were statistically equivalent (p>0.05) in the uninfected, Cry5B, and mebendazole treated groups (FIG. 10, bottom light panel).

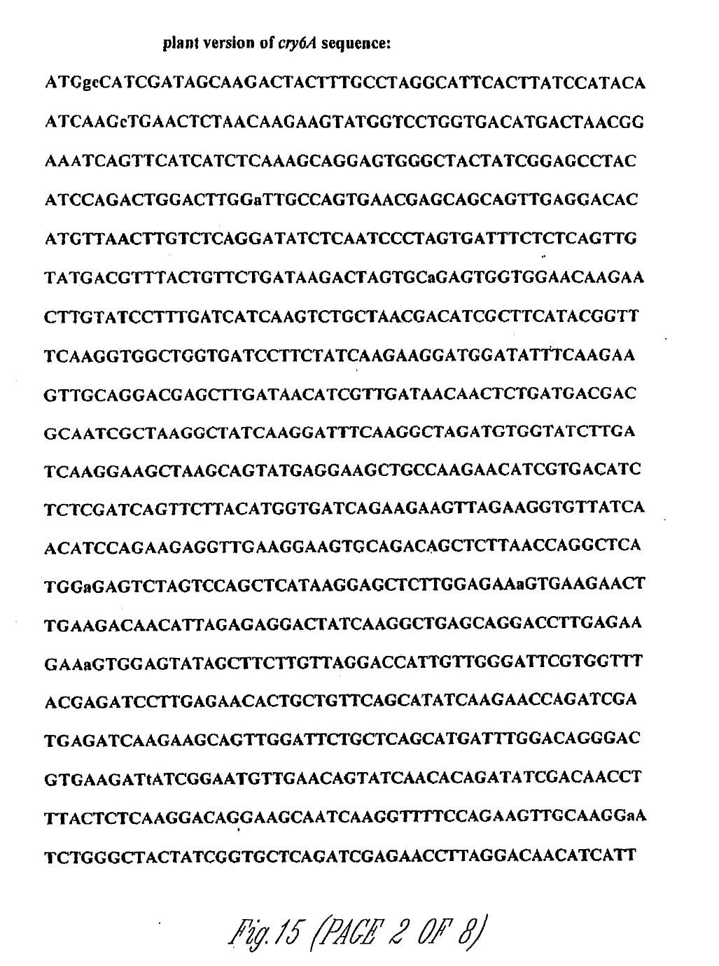

Cry5B treatment reduces fecal egg excretion and intestinal worm burden in hookworm infected hamsters. It was observed that parasite eggs can be detected in the feces of hamsters infected with A. ceylanicum as early as day 14 PI, with egg counts increasing sharply over the next several days. In the buffer treated animals, fecal egg counts progressed from a mean of 917+/−84 eggs per gram (epg) feces on day 16 PI to 1800+/−167 epg by day 19 PI (FIG. 11A). In contrast, hamsters treated with Cry5B excreted 217+/−84 epg at day 16 and 350+/−117 epg on day 19, a reduction of 76% (p=0.005 vs. buffer-treated group) and 81% (p=0.003), respectively. Although day 16 and day 19 epg in the Cry5B-treated group were higher than in the mebendazole-treated hamsters, this difference was not statistically significant.

To determine if the therapeutic effects of Cry5B treatment correlate also extend to a reduction in intestinal parasite burden, animals were sacrificed at day 22 and the number of adult worms counted. As shown in FIG. 11, the buffer-treated animals harbored a mean of 39.8 adult worms (range: 26-69). The animals in the Cry5B treatment group harbored significantly fewer adult worms (mean 4.5, range 0-23), which represented an 89% reduction in mean worm burden compared to the buffer-treated group (p<0.0001). Of note, more than half of the 45 total worms in the Cry5B-treated group were recovered from a single hamster. Moreover, 3 of the Cry5B-treated animals were cured of their infection, while a single worm was found in 3 additional animals in this treatment group. Despite higher mean worm burdens in the Cry5B treatment group compared to the mebendazole-treated animals (4.5 vs 0), this difference was not statistically significant (p ˜0.28).

### Discussion

The data presented here demonstrated that Cry5B, a purified crystal protein from Bt, is highly active against multiple life cycle stages of the human and animal hookworm parasite A. ceylanicum. Feeding of Cry5B to adult and larval hookworm stages results in significantly impaired nematode motility, decreased egg release, and morphological pathologies. Most important, the results demonstrate that oral administration of Cry5B to hookworm-infected hamsters results in significant therapeutic effects comparable to that of a standard anthelminthic, mebendazole. Namely, Cry5B administered orally to hookworm-infected hamsters results in recovery of weight loss, recovery of hemoglobin levels, a >5 fold reduction in fecal egg excretion, and a nearly 10 fold reduction in intestinal worm burden.

Previous data have pointed to the potential of crystal proteins against animal parasitic nematodes. For example, E. coli expressing Cry5B are toxic to the free-living stages of the rodent parasite Nippostongylus brasiliensis (Wei et al., 2003), and spore-crystal lysates from two Bt strains were found to be toxic to three nematodes of veterinary importance using in vitro assays. In the latter study, one of the strains also expressed Cry5B protein, although interpretation of these findings is complicated by the fact that each of the strains expressed multiple Cry proteins and that Bt spores were included in the assays. The results herein demonstrate that a purified crystal protein, independent of spores or any other factors made by Bt, are toxic to a human parasitic nematode both in vitro and in vivo.

The data presented here are also consistent with a similar mechanism of Cry5B action in hookworm and in C. elegans. Both nematodes express glycolipid receptors, and the sensitivities to Cry5B of various stages (larval and adult) and phenotypes (e.g., egg production) are very similar between the two organisms. Furthermore, as expected from studies in C. elegans and from the accepted mechanism of action of Cry proteins in general (de Maagd et al., 2001) and ingestion of the toxin is required for toxicity against hookworm since neither A. ceylanicum embryos nor non-feeding infectious L3 stage larvae are susceptible to the toxin.

The most striking observation is that orally administered purified Cry5B is highly effective at reducing the clinical sequelae of hookworm infection, namely anemia and growth delay. These studies utilized the hamster model of A. ceylanicum, a well characterized system for investigations of hookworm pathogenesis and host-parasite interactions (Bungiro et al., 2001; Held et al., 2006; Cappelo et al., 2003). Indeed, these studies indicate that the hamster model of A. ceylanicum appears to be an efficient means of characterizing anthelminthic effects of Cry proteins both in vitro and in vivo. Animals treated with three orally administered doses of Cry5B showed statistically significant improvements in weight and blood hemoglobin levels, along with reductions in worm burdens (as measured by fecal egg excretion and intestinal worm recovery) compared to control hamsters receiving buffer alone. The three day orally administered treatment regimen (Cry5B or mebendazole) is consistent with recommendations for treatment of individuals with intestinal nematode infections using benzimidazole anthelminthics, although community based programs often utilize single dose therapy (deSilva et al., 2003). Of note, no evidence of toxicity attributable to Cry5B was noted in any of the treated animals.

Despite the previously raised concerns that gastric acid would reduce the efficacy of orally administered Bt toxin (Kotzke et al., 2005), data from the present study using oral administration show that the effect of Cry5B was comparable to that seen with mebendazole, as measured by improvements in weight and blood hemoglobin concentrations of treated animals compared to controls. However, it is worth noting that Cry5B was less effective at eliciting complete cure of hookworm infection in the animal study. The degree to which this result reflects a fundamental difference in susceptibility of A. ceylanicum to Cry5B and mebendazole, or in fact suggests a degree of inactivation of the toxin during intestinal transit, is currently being investigated.

Anthelminthic chemotherapy remains the cornerstone of current control measures for human and animal STN infections (deSilva et al., 2003; Horten, 2003). Periodic deworming of school age children has been recommended to improve growth and nutritional status, while treatment of women infected with STNs during pregnancy may reduce the prevalence of anemia and improve birth outcomes (Crompton et al., 2002). Advantages of currently available benzimidazole anthelminthics, e.g., mebendazole and albendazole, include their broad spectrum of activity against all major species of STN, as well as their favorable safety profile and low cost (deSilva, 2003; Horton, 2003; Grover et al., 2001). However, evidence suggests that frequent deworming is necessary to achieve lasting benefit for school age children, which may have already led to the emergence of resistance in human isolates, particularly hookworm, from endemic communities. Repeated use of anthelminthics has already been associated with widespread resistance in livestock, dramatically reducing the efficacy of these agents in many veterinary settings.

With the potential emergence of benzimidazole resistance in communities where anthelminthics have been used extensively, the development of new chemotherapeutic agents that are safe, effective against a broad spectrum of human parasites, and inexpensive may prove essential in order to meet the WHO goals for global control of morbidity due to STN infections (Savioli et al., 2004; Savoili et al., 2005; Wito, 2000). Bt Cry proteins, including Cry5B, meet these criteria and, as pesticides, have an unprecedented track record of vertebrate safety (Betz et al., 2000). These studies define a potentially new class of anthelminthics for treatment and/or prevention of human parasitic nematode infections in billions of people at risk. Furthermore, the data presented here establish a compelling rationale for further pre-clinical development of Bt Cry toxins for therapeutic use.

The purified recombinant Cry5B protein is highly active against the hookworm Ancylostoma ceylanicum, a parasitic nematode for which humans are permissive hosts. Using in vitro culture methods, it was demonstrated that Cry5B targets multiple stages of hookworm development, including the intestinal bloodfeeding stage. It was also demonstrated that oral administration of purified Cry5B to hamsters infected with A. ceylanicum provides significant benefit in vivo, effecting a cure of the hookworm infection comparable to that achieved with mebendazole. Taken together, these data suggest that Bt Cry proteins are not only useful for control of insect pests that eat crops and transmit disease, but also have tremendous potential for treatment of parasitic nematode infections of humans and other mammals.

### Example V

### Experiments with Swiss Mice

In this experiment, eight female Swiss-Webster mice from Hilltop Laboratories were used as well as eight male mice. The mice were immunosuppressed with dexamethasone in the drinking water, and given about 500 eggs of T. muris per os. The mice were maintained with dexamethasone in the drinking water with standard laboratory rodent chow. One male mouse died of unknown causes.

Nine days post-infection, four male and four female mice were euthanized and the cecum and large intestines inspected for nematodes. The procedure followed was derived in part from Nippostrongylus work and the techniques in Campbell (1963) and Wakelin (1967). Basically, the cecum and large intestine including the rectum are removed from the mouse, and slit open and immersed in 0.15 M NaCl in a petri dish. Each organ system is placed in separate petri plates and kept at 37° C. for 1-2 hours. The plates are transferred to 5° C. overnight. During the examination, the mucosa is removed from the organs by scrapping with bent-tip forceps. Both saline and shredded mucosa are examined for the presence of nematodes which are counted and transferred to 10% formalin in 0.5 M NaCl. These nematodes are used for length measurements.

No nematodes were detected in the samples. Because of the negative findings, all samples were inspected twice. However, since all the subsequent mice examined were infected, the nematodes were overlooked which according to Panesar and Croll (1980) are completely embedded in the mucosa until day 15 and are less than 2 nm long (Wakelin, 1967).

115 days post-infection, the remaining mice were euthanized and necropsied as above. The presence of nematodes was detected in all three remaining male and four female mice. Because of the abundance of nematodes, the samples were stored in 10% formalin and 0.15 M NaCl. The samples are centrifuged, washed once with saline, recentrifuged and examined as described for the fresh samples. Four mice (three females and one male) have been examined with 102 to 339 (average ˜224) nematodes recovered. The nematodes are minute with an estimated length of about 2 mm. Pinworms were present in the male mice.

Experiments with AKR mice

Male AKR mice (4 weeks old) were obtained from Jackson Laboratories. Most of the current papers dealing with T. muris use AICR mice which are relatively susceptible to T. muris which persists for at least 42 days (see Cliffe et al., 2005).

The mice (average mass=19.5 g) were infected per os with about 500 eggs of T. muris. Dexamethasone was not used since these mice are susceptible to T. muris. The mice were maintained in groups of three under standard animal room conditions until 23 days post-infection. At this time the mice, two cages, 6 mice, were designated controls and each received 0.1 mL of citrate buffer. During anesthesia, two control mice were killed accidentally. The mice in the remaining two cages received one milligram of Cry5B in citrate buffer. Treatment was repeated two more times for a total of three treatments on days 23, 24, and 25 post-infection.

28 days post-infection, the mice were euthanized and posted as described above.

The results are shown in the following table:

| | Cecum | Colon | Total |
|---|---|---|---|
| Control | 1 | 66 | 67 |
| | 15 | 57 | 72 |
| | 1 | 1 | 2 |
| | 2 | 20 | 22 |
| Average | 4.75 | 36 | 40.75 |
| CTP | 3 | 4 | 7 |
| | 2 | 0 | 2 |
| | 1 | 0 | 1 |
| | 0 | 2 | 2 |
| | 0 | 12 | 12 |
| | 1 | 4 | 5 |
| Average | 1.167 | 3.667 | 4.833 |

Nematode counts are shown for the cecum, colon (large intestine and rectum), and the total number of worms recovered for each mouse. In the control group, the recovery ranged from 2 to 72 nematodes for a mean of 40.75 for four control mice. Two of the controls were lost during the treatment phase of the experiment. In the experimental group treated with Cry5B, the nematode recovery ranged from 1 to 12 nematodes with a mean of 4.83 from six mice. The unequal variances in the data arrays vitiated the use of the student t-test and the nonparametric statistic, the Mann-Whitney U-test.

### U Test Results

| n1 | n2 | U | P (two-tailed) | P (one-tailed) |
|---|---|---|---|---|
| 6 | 4 | 20.0 | 0.1142858 | 0.0571429 |

| | | |
|---|---|---|
| normal approx z = 1.70561 | 0.0880816* | 0.0440408* |

*These values are approximate.

This analysis was done with the use of the on-line Mann-Whitney U-test on the “eatwoms” website. Although the two samples are not significantly different (P >=0.05) with a two-tailed test, they are significantly different with a one-tail test. This complies with the null hypothesis for such data where it was hypothesize that the treatment will not reduce the worm count.

After the counting, the nematode length is determined for comparison with the growth curve in Wakelin (1967). The worms appear to be more abundant in the dexamethasone-treated mice (Swiss-Webster).

As for the AKR nematodes, the nematode lengths are determined as another measure of the effect of Cry5B. Presumably, length increases are inhibited in the presence of Cry5B. Unfortunately, the sample size is relatively small.

### Example VI

Truncated Cry5B was expressed in tomato hairy roots and found to inhibit the production of root-knot nematode (M. incognita) progeny. Thus, truncated Cry5B can be used to control plant parasitic nematodes. The data from two experiments were as follows:

| | average total eggs | average total eggs |
|---|---|---|
| control 70S line 70-7 | 3312.5 (4 plates) | 16920 (5 plates) |
| control 70S line 70-14 | 6861.1 (3 plates) | 22200 (5 plates) |
| Cry5B line 5B-32 | 937.5 (4 plates) | 10040 (5 plates) |
| Cry5B line 5B-31 | 1983.3 (4 plates) | 5980 (5 plates) |
| Cry5B line 5B-15 | 1111.1 (3 plates) | 6700 (5 plates) |

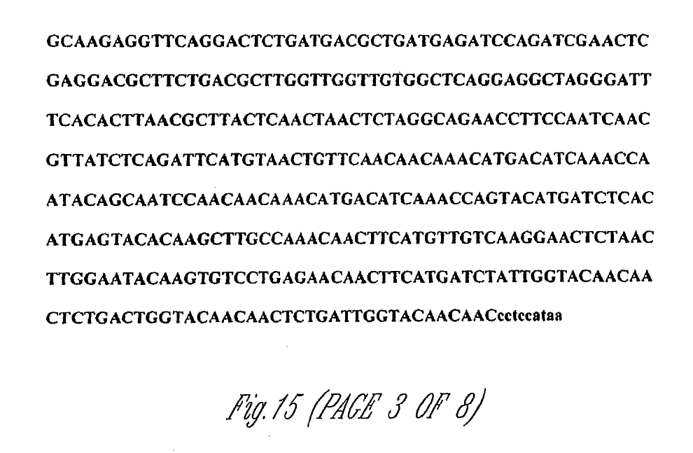

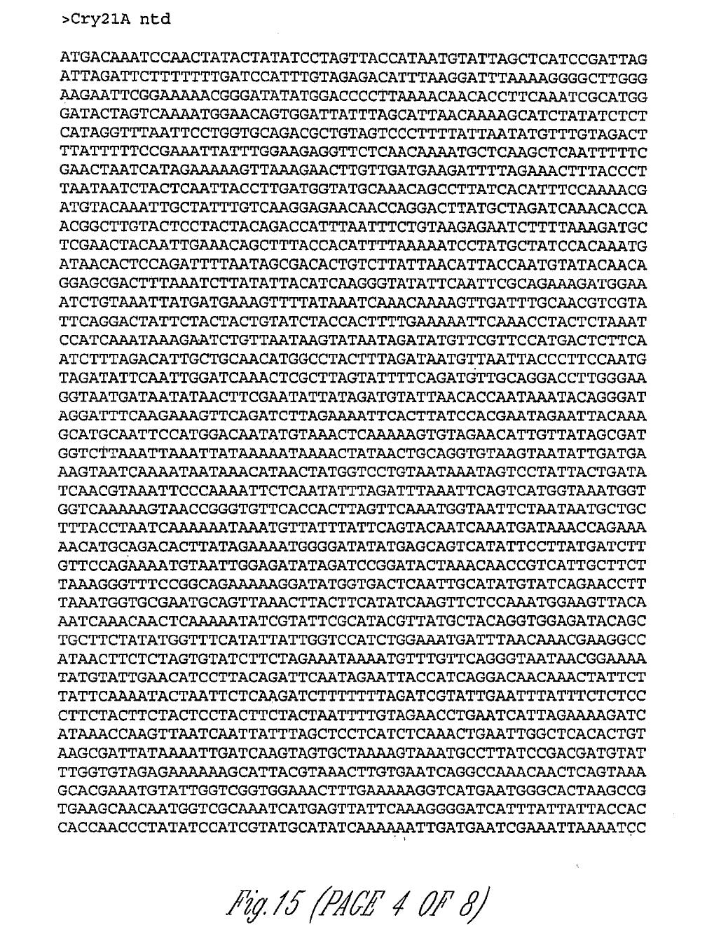

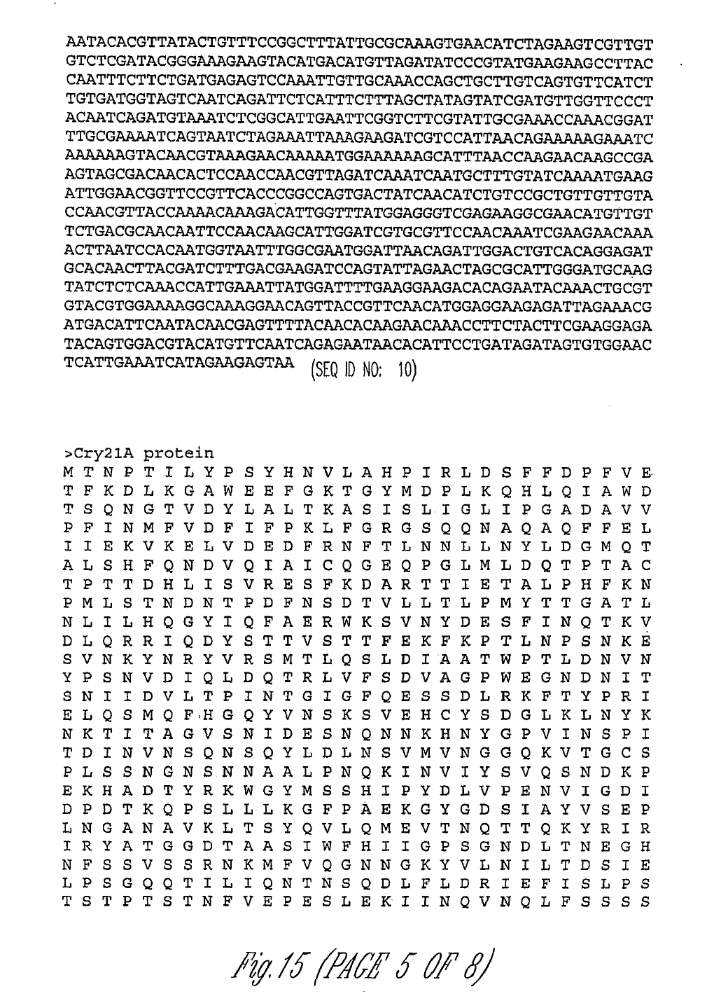

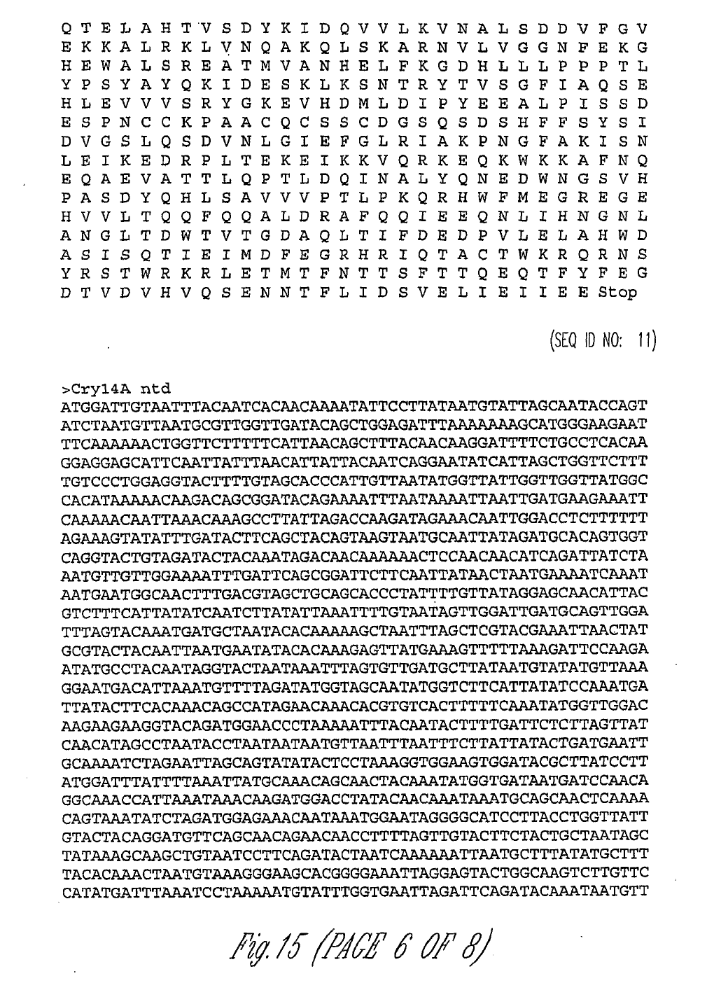

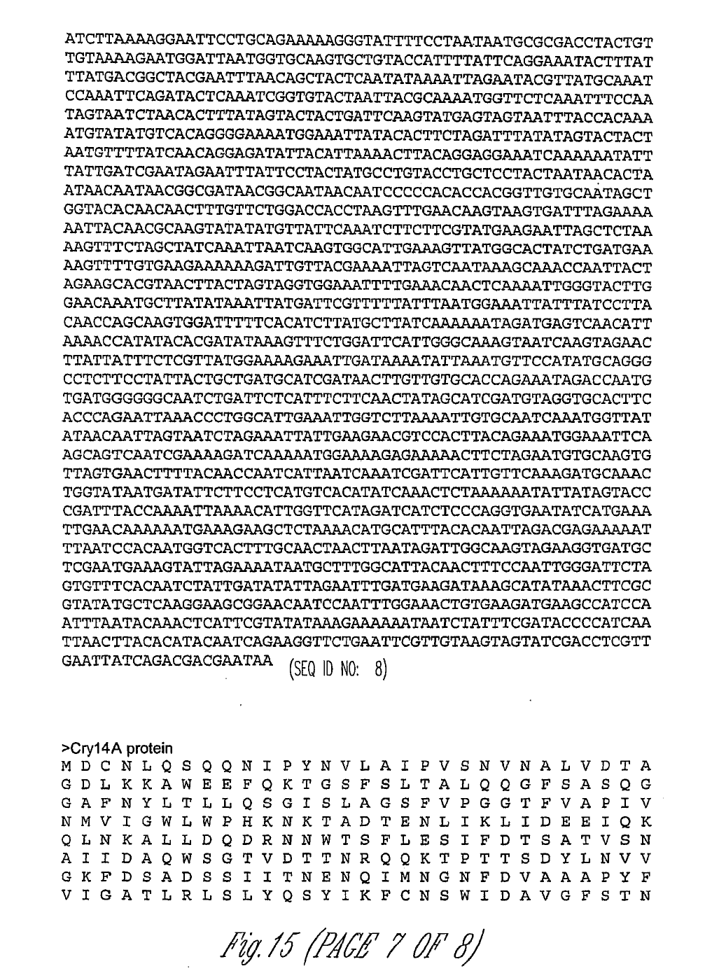

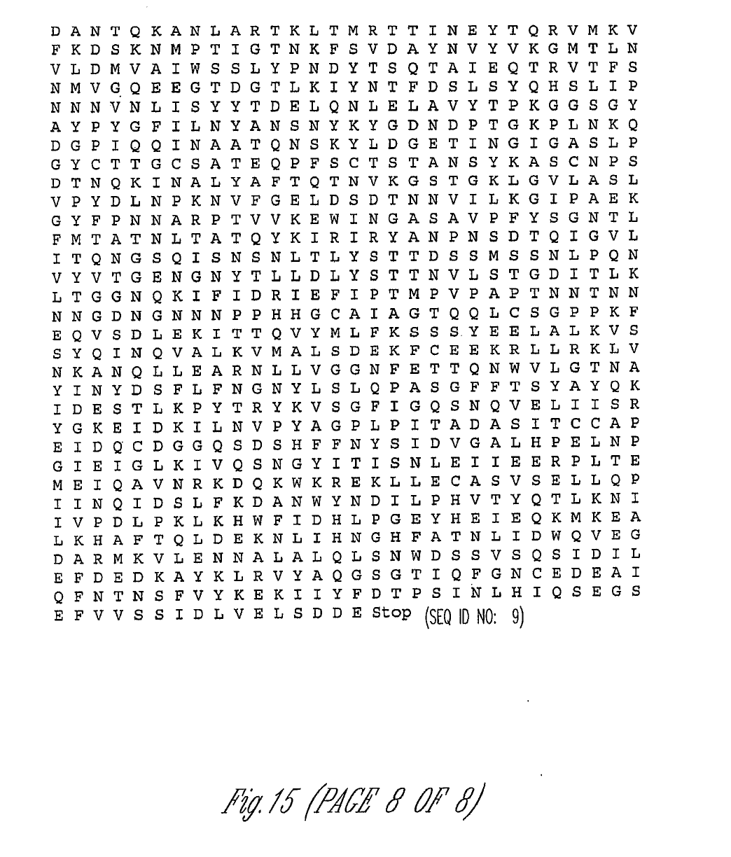

Cry14A and Cry21A will likely also be toxic to human parasitic nematodes, e.g., hookworm, as they are toxic to the other nematodes that Cry5B is toxic to, including the rodent parasite Nippostrongylus. The toxicity of truncated Cry5B, Cry14A, and Cry21A against C. elegans is shown in FIGS. 12-13.

### REFERENCES

Albonico et al., Ann. Trop. Med. Parasitol., 96:717 (2002).
Albonico et al., Int. J. Parasitol., 35:803 (2005).
Albonico et al., Int. J. Parasitol., 34:1205 (2004).
Albonico, Acta Trop., 86:233 (2003).
Areekul et al., J. Med. Assoc. Thai., 53:190 (1970).
Atkinson et al., Transgenic Res., 13:135 (2004).
Betz et al., Regul. Toxicol. Pharmacol., 32:156 (2000).
Blasingame & Patel, In: Proceedings of Beltwide Cotton Conference 252-253 (National Cotton Council, Nashville, Tenn.; 2003).
Bockenhoff & Grundler, Parasitology, 109:249 (1994).
Brownbridge et al., J. Invertebr. Pathol., 48:216 (1986).
Bungiro et al., Am. J. Trop. Med. Hyg., 73:915 (2005).
Bungiro et al., Infect. Immun., 72:2203 (2004).
Bungiro et al., J. Infect. Dis., 183:1380 (2001).
Bustos et al., Plant Cell, 1:839 (1989).
Cai et al. Science, 275:832 (1997).
Callis et al., Plant Physiol., 88:965 (1998).
Campbell, J. Parasitology, 49: 628 (1963).
Cappello et al., J. Parasitol., 89:S158 (2003).
Chan, Parasitol. Today, 13:438 (1997).
Chandrawathani et al., Trop. Anim. Health Prod., 35:17 (2003).
Chowdhury et al., Am. J. Trop. Med. Hyg., 21:300 (1972).
Chu et al., Infect. Immun., 72:2214 (2004).
Cliffe et al., Science, 308:1463 (2005).
Coles et al., Vet. Parasitol., 129:345 (2005).
Coles, Res. Vet. Sci., 78:99 (2005).
Crompton et al., Acta Trop., 86:121 (2003).
De Clercq et al., Am. J. Trop. Med. Hyg., 57:25 (1997).
de Maagd et al., Trends Genet., 17:193 (2001).
De Rocher et al., Plant Physiol., 117:1445 (1998).
de Silva et al., Trends Parasitol., 19:547 (2003).
de Silva, Acta Trop., 86:197 (2003).
Debro et al., J. Bacteriol., 165:258 (1986).
Diehn et al., Plant Physiol., 117:1433 (1998).
El-Alfy & Schlenk, Toxicological Sciences, 68:381 (2002).
Pederici, J. Invertebr. Pathol., 89:30 (2005).
Ferrandiz et al., Science, 289:436 (2000).
Garside et al., Parasitology, 98:283 (1989).
Geerts et al., Trop. Med. Int. Health, 6:915 (2001).
Goverse et al. Physiological & Molecular Plant Pathology 52:275 (1998).
Griffitts et al., Science, 293:860 (2001).
Griffitts et al., Science 307:922 (2005).
Grover et al., Trop. Gastroenterol., 22:180 (2001).
Held et al., Infect. Immun., 74:289 (2006).
Hong et al., Plant Physiology, 119:1165 (1999).
Horton, Trends Parasitol., 19:527 (2003).
Hwang et al., Plant Cell, 12:1319 (2000).
Jame, C. in ISAAA Briefs No. 32 Ithaca, N.Y.; 2004.
Jung & Wyss, Applied Microbiology & Biotechnology 51:439 (1999).
Kotze et al., Int. J. Parasitol., 35:1013 (2005).
Kuhlemeier et al., Plant Cell, 1:471 (1989).
Lifshitz & Gavrilov, J. Toxic.—Clin. Toxicol., 38:799 (2000).
Lochnit et al., Biol. Chem., 381:839 (2000).
Marcotte et al., Plant Cell, 1:969, 1989).
Marroquin et al., Genetics, 155:1693 (2000).
McKellar et al., Trends Parasitol., 20:456 (2004).
Med Lett Drugs Ther., 1189:1 (2004).
Mertz et al., J. Am. Vet. Med. Assoc., 226:779 (2005).
Molyneux et al., PLoS Med., 2:e336 (2005).
Naimov et al., Appl. Environ. Microbiol., 67:5328 (2001).
Panesar & Croll, Int'l J. Parasitology, 10:261 (1980).
Plovie et al. Nematology, 5:831 (2003).
Qaim & Zilberman, Science, 299:900 (2003).
Reynoldson et al., Acta Trop., 68:301 (1997).
Ristaino & Thomas, Plant Disease, 81:965 (1997).
Roshal et al., EMBO J., 6:1155 (1987).
Saathoff et al., BMC Infect. Dis. 4:27 (2004).
Sambrook et al., in, Vol. 2 9.31-39.57 (Cold Spring Harbor Laboratory Press, NY; 1989).
Sasser & Freckman, Edn. Veech, J. A. Dickerson, D. W. 7-14 (Society of Nematologists, Hyattsville, Md., USA; 1987).
Savioli et al., Lancet, 365:1520 (2005).
Savioli et al., Nat. Rev. Microbiol., 2:618 (2004).
Savioli et al., Parasitol. Int., 53:103 (2004).
Schemthaner et al., EMBO J., 7:1249 (1988).
Speijer & De Waele, Vol. 1997, Edn. http://www.inibap.org/publications/technicalguidelines/guideline_eng.htm (International Network for the Improvement of Banana and Plantain, 1997).
Urbani et al., Acta Trop., 86:215 (2003).
Urwin et al., Molecular Breeding 6:257 (2000).
Urwin et al., Molecular Plant-Microbe Interactions, 15:747 (2002).
Urwin et al., Plant Journal, 12:455 (1997).
Urwin et al., Plant Journal, 8:121 (1995).
Urwin et al., Planta, 204:472 (1998).
Utzinger et al., Expert Opin. Pharmacother., 5:263 (2004).
von Samson-Himmelstjerna et al., Vet. Parasitol., 132:223 (2005).
Wakelin, Parasitology, 57:515 (1967)
Wei et al., Proc. Natl. Acad. Sci. USA, 100:2760 (2003).
Whalon et al., Arch. Insect. Biochem. Physiol., 54:200 (2003).
WHO (2000) Report by secretariat. 27 Oct. 2000. Division of Communicable Diseases, EB 107/31.
Williamson & Hussey, Plant Cell, 8:1735 (1996).
Wolstenholme et al., Trends Parasitol., 20:469 (2004).
Wu et al., FEBS Letters, 473:227 (2000).
Yoshida et al., Am. J. Trop. Med. Hyg., 17:378 (1968).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A transgenic plant comprising a recombinant DNA sequence operably linked to a promoter functional in plant cells, wherein the recombinant DNA sequence encodes a Bacillus thuringiensis (Bt) crystal (Cry) protein, wherein the Cry protein is a Cry6 or Cry5 protein, and wherein the Cry6 or Cry5 protein is expressed so as to impart nematode resistance to the transgenic plant.

2. The transgenic plant of claim 1 wherein the protein is expressed in the roots of the transgenic plant.

3. The transgenic plant of claim 1 wherein the plant is a dicot.

4. The transgenic plant of claim 1 wherein the codons of the DNA sequence have been altered to increase its expression in the cells of a plant, such as by including codons preferred by the plant, or by removing sequences such as polyA sites, splice sites or mRNA destabilization sequences, or both.

5. The transgenic plant of claim 1 wherein the DNA sequence has been truncated, so that a truncated Cry6 or Cry5 protein is expressed.

6. The transgenic plant of claim 5 wherein the truncation yields a nematode-toxic Cry6 protein that retains amino acid residues corresponding to residues 1 to 382 or 1 to 384 of Cry6A (SEQ ID NO:3).

7. The transgenic plant of claim 5 wherein the truncation yields a nematode-toxic Cry6 protein that retains amino acid residues corresponding to residues 1 to 386 of Cry6A (SEQ ID NO:3).

8. The transgenic plant of claim 1 wherein the Cry6 protein contains at least one substitution at one or more positions corresponding to residue 19, 52, 62, 99, 121, 138, 147, 164, 170, 192, 205, 237, 249 or 319 in SEQ ID NO:3.

9. The transgenic plant of claim 5 wherein the truncation yields a truncated Cry5 protein that retains amino acid residues corresponding to residues 1 to 769, 1 to 714, 1 to 698, or 1 to 933 of SEQ ID NO:2.

10. The transgenic plant of claim 5 wherein the truncation yields a truncated Cry5 protein that retains amino acid residues corresponding to residues 1 to a residue including residue 698, 718, 769 or 933, or any integer between residue 698 and 933, of SEQ ID NO:2.

11. The transgenic plant of claim 1 wherein the Cry5 protein has a substitution at a position corresponding to position 407 or 663 of SEQ ID NO:2

12. The transgenic plant of claim 1 wherein the promoter is the CaMV 35S promoter.

13. The transgenic plant of claim 1 wherein the DNA sequence encodes a 54 kDa Cry6A protein.

14. The transgenic plant of claim 1 which is a monocot.

15. The transgenic plant of claim 1 which is a dicot.

16. A pharmaceutical composition comprising one or more isolated crystal proteins in an amount effective to inhibit or treat helminth infection in a mammal and a pharmaceutically acceptable carrier, wherein the isolated crystal protein is Cry5B, Cry21A, or Cry14A.

17. The composition of claim 16 which comprises two or more of isolated Cry5B, Cry21A and Cry14A.

18. A method to inhibit or treat parasitic worm infection in a vertebrate, comprising: administering to a vertebrate an effective amount of one or more Cry proteins.

19. The method of claim 18 wherein the vertebrate has hookworm.

20. The method of claim 18 wherein the vertebrate is a human.

21. The method of claim 18 wherein the vertebrate is a mammal.

22. The method of claim 18 wherein the administration is oral.

23. The method of claim 18 wherein the crystal protein comprises Cry5B.

24. The method of claim 18 wherein the crystal protein comprises Cry21A.

25. The method of claim 18 wherein the crystal protein comprises Cry14A.

26. The method of claim 18 wherein the vertebrate is administered a transgenic plant food product comprising the crystal protein.

27. An isolated nucleic acid molecule comprising a nucleic acid segment encoding a truncated Cry5, Cry6, Cry14 or Cry21 protein having nematicidal activity, wherein the truncated protein has at least 95% amino acid sequence identity to SEQ ID NO:2, SEQ ID NO:3, SEQ ID NO:9 or SEQ ID NO: 11.

28. The nucleic acid molecule of claim 27 wherein the Cry protein is truncated at the N-terminus, C-terminus or both.

29. The nucleic acid molecule of claim 27 wherein the nucleic acid segment encodes a truncated Cry5 or Cry6 that has at least 90% nucleic acid sequence identity to SEQ ID NO:7 or SEQ ID NO:1.

30. The nucleic acid molecule of claim 27 wherein the nucleic acid segment encodes a truncated Cry5B or Cry6A that has one or more amino acid substitutions relative to SEQ ID NO: or SEQ ID NO:3.

31. The nucleic acid molecule of claim 27 wherein the truncated Cry6 has a substitution at one or more positions corresponding to residue 19, 52, 62, 99, 121, 138, 147, 164, 170, 192, 205, 237, 249 or 319 in SEQ ID NO:3.

32. The nucleic acid molecule of claim 27 wherein the truncated Cry5 has a substitution at a position corresponding to position 407 or 663 in SEQ ID NO:2.

33. An isolated nucleic acid molecule comprising a nucleic acid segment encoding a Cry5 or Cry6 protein with one or more substitutions and having nematicidal activity, wherein the Cry5 or Cry6 protein has at least 95% identity to SEQ ID NO:2 or SEQ ID NO:3.

34. The nucleic acid molecule of claim 33 wherein Cry6 is truncated at the N-terminus, C-terminus or both.

35. The nucleic acid molecule of claim 33 wherein Cry5 is truncated at the N-terminus, C-terminus or both.

36. The nucleic acid molecule of claim 33 wherein the Cry6 has a substitution at one or more positions corresponding to residue 19, 52, 62, 99, 121, 138, 147, 164, 170, 192, 205, 237, 249 or 319 in SEQ ID NO:3.

37. The nucleic acid molecule of claim 33 wherein the Cry5 has a substitution at a position corresponding to position 407 or 663 in SEQ ID NO:2.

38. A polynucleotide which hybridizes under stringent conditions to SEQ ID NO:1, SEQ ID NO:7 or the complement thereof.
